(19) United States
(12) Patent Application Publication
(10) Pub. No.: US 2010/0189657 A1
(43) Pub. Date: Jul. 29, 2010

(54) INTRAMOLECULARLY QUENCHED FLUOROCHROME CONJUGATES AND METHODS OF USE

(75) Inventors: Ralph Weissleder, West Peabody, MA (US); Ching-Hsuan Tung, Wayland, MA (US); Yongdoo Choi, Gwangju (KR)

Correspondence Address: EDWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/293,675

(22) PCT Filed: Mar. 20, 2007
(86) PCT No.: PCT/US07/07289

§ 371 Date: Apr. 15, 2010

Related U.S. Application Data
(60) Provisional application No. 60/783,959, filed on Mar. 20, 2006.

(51) Int. Cl. A61K 49/00; C07K 2/00; C07H 21/00; C08G 69/48; C08G 63/91; C07K 14/76; C07K 16/00; A61K 38/02; A61P 35/00

(52) U.S. Cl. 424/9.6; 530/300; 536/22.1; 536/123.1; 525/420; 525/55; 530/362; 530/391.3; 525/418; 525/450; 514/2

(57) ABSTRACT

The invention provides a quenched fluorochrome conjugate and methods of use thereof in the detection and treatment of disorders characterized by unwanted cellular proliferation including cancer.

### CROSS-REFERENCE TO RELATED APPLICATIONS/PATENTS & INCORPORATION BY REFERENCE

This application claims priority to U.S. Provisional Application Ser. No. 60/783,959, filed on Mar. 20, 2006, the contents of which are incorporated herein by reference.

Each of the patent applications, patents and other references and documents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Photodynamic therapy (“PDT”) employs photoactivatable compounds known as photosensitizers to selectively target and destroy cells. Therapy involves delivering visible light of an appropriate wavelength to excite a photosensitizer molecule to its excited singlet state. This excited state can then undergo intersystem crossing to the slightly lower energy triplet state, which can then react further by one or both of two pathways, known as Type I and Type II photoprocesses (Ochsner (1997) J Photochem Photobiol B 39:1-18). The Type I pathway involves electron transfer reactions from the photosensitizer triplet to produce radical ions that can then react with oxygen to produce cytotoxic species such as superoxide, hydroxyl and lipid derived radicals. The Type II pathway involves energy transfer from the photosensitizer triplet to ground state molecular oxygen (triplet) to produce excited state singlet oxygen, which can then oxidize biological molecules such as proteins, nucleic acids and lipids, and lead to cytotoxicity.

In practice, a photosensitizer, such as a porphyrin derivative, is administered to a subject and retained in the target tissue(s) of the subject, followed by laser irradiation to cause selective destruction of the target tissues. This approach utilizes the selectivity for proliferative tissues and the photosensitivity associated with a porphyrin derivative to destroy the tissue. Unfortunately, distribution to target tissues is often not sufficiently selective to prevent accumulation in normal tissues and, therefore, many photodynamic compositions cause temporary photosensitivity as an undesirable side effect when administered to the human body.

Under the circumstances, a patient treated with a photodynamic composition is required to stay in the dark for a long period of time until the photodynamic composition is completely excreted from the body so that normal cells are not damaged by the photosensitizing action of the photodynamic composition accumulated in normal tissues. However, because the photodynamic composition shows a slow excretion rate from normal tissues, it sometimes causes photosensitivity to last for more than six weeks. In addition, PDT may have problems with transmission of the light irradiated by laser through tissues. That is, some PDT compositions have a longest wavelength absorption end at 630 nm and a molar absorption coefficient as small as 3,000. Because there are many components present in a living body which prevent the transmission of light, such as oxyhemoglobin and water, the light with wavelength of 630 nm exhibits a poor transmission through tissues, which cannot sufficiently reach to deep sites, thus, PDT is particularly suited for treating disorders developing in the surface layers of 5 to 10 mm depth. The wavelength which is least damaging by the light absorption to the components in a living body is in a range of 650 to 750 nm, therefore, photosensitizers for PDT having the longest wavelength absorption end within such a range are especially desirable.

Targeted phototoxic probes are valuable tools in PDT (van Dongen, G. A. M. S.; Visser, G. W. M.; Vrouenrasts, M. B. Advanced Drug Delivery Rev. 2004, 56, 31-52).

The efficiency of such probes can be improved if their fluorescence emission can be turned on only at the target site. Recently enzyme targeting has been used to reveal tissue specific molecular information by imaging (Tung, C. H. Biopolymers 2004, 76, 391-403). One strategy for the design of such imaging probes is by incorporation of a molecular switch where the fluorochromes in the probe are quenched efficiently. Similarly, PDT can be designed using the same approach. A variety of peptidic (Pham, W.; Choi, Y. D.; Weissleder, R.; Tung, C. H. Bioconjugate Chem. 2004, 15, 1403-1407), polymeric (Weissleder, R.; Tung, C. H.; Mahmood, U.; Bogdanov, A. Nature Biotechnol. 1999, 17, 375-378), and nanospheric (Josephson, L.; Kircher, M. F.; Mahmood, U.; Tang, Y.; Weissleder, R. Bioconjugate Chem. 2002, 13, 554-560) scaffolds have been used to conjugate fluorescent dyes. Macromolecular polymer- and nanosphere-based systems have been shown to work exceedingly well because they provide a platform for efficient quenching and can be delivered readily to tumors because of the enhanced permeability and retention (EPR) effect (Maeda, H. Adv. Enzyme Regul. 2001, 41, 189-207). Such macromolecular systems, however, have certain drawbacks, including solubility and toxicity concerns. Most polymer- or nanosphere-based systems have polydispersity and heterogeneity associated with them making the precise characterization of the probe difficult or ambiguous. Additionally, neither the specific site nor the extent of conjugation can be easily controlled in such systems. Finally, some of these macromolecular probes may get retained in the non-target tissues for extended periods of time due to their long circulation time, and slow blood clearance.

In addition, in many clinical settings it is desirable to have the ability to combine or make use of both imaging and treating a disease simultaneously. In the specific case of PDT, it would be advantageous to able to detect and locate the disease prior to activating the phototherapy. It would also be adventageous to be able to monitor the release or delivery of the photosensitzers at the site of disease.

As mentioned above, photosensitizers currently used for PDT have various defects and, therefore, development of new agents without such defects is strongly desired. Thus, there exists a need in the art for improved methods of detection and treatment of disease such as unwanted cellular proliferation (e.g., in cancers and tumors) with minimal toxicity and sensitivity to light.

### SUMMARY OF THE INVENTION

The instant invention features fluorochrome conjugates, which are initially quenched and, therefore, insensitive to light prior to protease-mediated degradation at or within a target cell. Following administration, the quenched conjugates are converted to active fluorochromes by proteases present either in the region of or within the target cell. Where the fluorochrome is a photosensitizer, local illumination generates cytotoxic singlet oxygen sufficient to kill or damage target cells. Advantages over traditional photodynamic approaches include minimal invasiveness, enrichment in a diseased tissue area, protease dependent tissue specificity, ability to image before, during and after treatment, and minimal side-toxicity due to the non-activated state of the photosensitzer conjugates in circulation.

In one aspect, the invention provides a fluorochrome conjugate comprising, a backbone, a protease cleavage site; and at least two fluorochromes, each covalently linked by a protease cleavage site to the backbone at quenching positions, wherein at least one fluorochrome is a photosentizer.

In another aspect, the invention provides a fluorochrome conjugate comprising, a dendrimer; a peptide wherein the peptide comprises a protease cleavage site; and at least two fluorochromes, each covalently linked to the dendrimer at optical-quenching positions.

In certain embodiments, the fluorochromes are photosensitizers.

In certain embodiments, the fluorochromes are near-infrared fluorochromes.

In certain embodiments, the fluorochromes have excitiation and emission maxima in range of about 500 nm to about 900 nm.

In other embodiments, the fluorochrome conjugate comprises a combination of photosenstizer fluorochromes and non-photosensitizer fluorochromes.

In other embodiments, the fluorochrome conjugate comprises a combination of photosenstizer fluorochromes and quencher.

In other embodiments, the invention provides a fluorochrome conjugate further comprising a spacer compound; wherein the spacer compound links the fluorochrome to the dendrimer.

In still other embodiments, the invention provides a fluorochrome conjugate further comprising at least one solubility enhancing group.

In still other embodiments, the invention provides a fluorochrome conjugate further comprising at least one targeting moeity.

In certain embodiments, the invention provides a fluorochrome conjugate, wherein the dendrimer is a branched polypeptide, a branched nucleic acid, a branched polyethyleneamine, a branched polysaccharide, a branched polyamidoamine, a branched polyacrylic acid, a branched polyalcohol or a branched synthetic polymer. In particular embodiments, the dendrimer is a STARBURST PAMAM (polyamidoamine) dendrimer, dense star dendrimer, non-dense star dendrimer, arborol dendrimer, self-immolative dendrimer, polypropyleneimine (PPI) dendrimer, phosphorous containing dendrimer, or a commercially available dendrimer, including but not limited to Polyester, POPAM, porphyrin-, podand-, organometallic-, or silicon-based dendrimers with various cascade architectures.

In further embodiments, the dendrimer is a branched polypeptide. In certain embodiments, the branched polypeptide comprises D or L amino acids or a combination thereof. In yet further embodiments, the branched polypeptide comprises polylysine. In certain embodiments, the branched polypeptide comprises poly-L-lysine.

In other embodiments, the branched polypeptide comprises albumin. In another embodiment, the branched polypeptide comprises multiple antigenic peptides. In certain embodiments, the branched polypeptide is an antibody or an antibody fragment.

In certain embodiments, the invention provides a fluorochrome conjugate, wherein the synthetic polymer is polyglycolic acid, polylactic acid, poly(glycolic-colactic) acid, polydioxanone, polyvalero lactone, poly-ε-caprolactone, poly(3-hydroxybutyrate), poly(3-hydroxyvalerate) polytartronic acid, poly aspartic acid, polyglutamic acid, or poly(β-malonic acid).

In other embodiments, the invention provides a fluorochrome conjugate, wherein the protease cleavage site has an amino acid sequence wherein the sequence is: RR, RRG, GPICFFRLG (SEQ. ID. NO. 1), HSSKLQG (SEQ. ID. NO. 2), PIC(Et)FF (SEQ. ID. NO. 3), HSSKLQ (SEQ. ID. No. 4), P(L/Q)G(I/L)AG (SEQ. ID. NO. 5), GVVQASCRLA (SEQ. ID. NO. 6) or KK. In certain embodiments, the protease cleavage site has an amino acid sequence of Leu-Arg.

In another embodiment, the invention provides a fluorochrome conjugate, wherein the protease cleavage site is cleaved by a protease wherein said protease is a cathepsin, matrix metalloproteinase (MMP), collagenase, gelatinase, stromelysin, caspase, viral protease, HIV protease, HSV protease, gelatinase, urokinase, secretase, endosomal hydrolase, endopeptidase, or Cytomegalovirus (CMV) protease. In a further embodiment, the cathepsin is Cathepsin B, Cathepsin D, Cathepsin H, Cathepsin K, Cathepsin L, and Cathepsin S.

In another embodiment, the invention provides a fluorochrome conjugate, wherein the spacer molecule is a peptide, oligopeptide, polysaccharide, a nucleic acid, or a synthetic cleavable moiety. In a further embodiment, the spacer molecule is a peptide. In another further embodiment, the peptide is comprised of glycine or β-alanine.

In other embodiments, the invention provides a fluorochrome conjugate, wherein the solubility enhancing group links the fluorochrome to the spacer compound. In certain embodiments, the solubility enhancing group links the fluorochrome to the protease cleavage site. In certain embodiments, the solubility enhancing group is polyethylene glycol (PEG), methoxypolyethylene glycol (MPEG), methoxy polypropylene glycol, copolymers of polyethylene glycol and methoxy polypropylene glycol, dextran, and polylactic-polyglycolic acid, polyethylene glycol-diacid, PEG monoamine, MPEG monoamine, MPEG hydrazide, MPEG imidazole, copolymers of polyethylene glycol, methoxypolypropylene glycol, or mixtures thereof. In a further embodiment, the solubility enhancing group is polyethylene glycol (PEG).

In another embodiment, the invention provides a fluorochrome conjugate that is a photosensitizer conjugate, wherein the photosenstizer is a chlorin (e.g., chlorin e6). In other embodiments, the photosenstizer is a porphyrin. In certain embodiments, the photosenstizer is rose bengal, a tetra pyrrole bacteriochlorin, hematoporphyrin, chlorin e6, tetraphenylporphyrin, porfimer sodium, phthalocyanine, naphthocyanine or benzoporphyrin.

In other embodiments, the photosensitizer and the solubility enhancing group associate to form a photosensitive moiety. In a further embodiment, the photosensitizer is rose bengal, a chlorin, a tetra pyrrole, or a porphyrin and the solubility enhancing group is polyethylene glycol (PEG), methoxypolyethylene glycol, methoxy polypropylene glycol, copolymers of polyethylene glycol and methoxypolypropylene glycol, dextran, polylactic-polyglycolic acid, and mixtures thereof. In certain embodiments, the photosensitive moiety comprises chlorin e6 and polyethylene glycol (e.g., Ce6PEG-1, Ce6PEG-2, or Ce6PEG-3).

In another aspect, the invention provides a fluorochrome conjugate comprising, a dendrimer; a peptide comprising a protease cleavage site; a solubility enhancing group; at least two fluorochromes, each independently and covalently linked to the dendrimer at optical-quenching positions; and one or more spacer compounds, wherein a spacer compound links a fluorochrome to the dendrimer. In certain embodiments, the photosensitizers are linked to the solubility enhancing group. In other embodiments, the solubility enhancing group is linked to a spacer compound. In other embodiments, the spacer compound is linked to the peptide. In other embodiments, the peptide is linked to a second spacer compound. In a further embodiment, the second spacer compound is linked to the dendrimer.

In another embodiment, the invention provides a fluorochrome conjugate, wherein the fluorochromes are linked to a solubility enhancing group; the solubility enhancing group is linked to a spacer compound; the spacer compound is linked to a peptide; the peptide is linked to a second spacer compound; and the second spacer compound is linked to the dendrimer.

In certain embodiments, the dendrimer is a branched polypeptide. In further embodiments, the branched polypeptide comprises poly-L-lysine. In a further embodiment, the protease cleavage site has an amino acid sequence of Leu-Arg. In still a further embodiment, the protease cleavage site is cleaved by a protease wherein said protease is Cathepsin B, Cathepsin D, Cathepsin H, Cathepsin K, Cathepsin L, or Cathepsin S.

In other embodiments, the solubility enhancing group is polyethylene glycol (PEG). In another embodiment, the photosenstizer is chlorin e6. In other embodiments, the spacer molecule is a peptide. In another embodiment, the peptide is comprised of glycine or β-alanine.

In another aspect, the invention provides a fluorochrome conjugate comprising: a polylysine dendrimer; at least one PEG solubility enhancing group; and at least two chlorin e6 molecules covalently linked through one or more spacers to the dendrimer at optical-quenching positions, wherein the spacers comprises a β-alanine and/or a cathepsin S enzymatic cleavage site.

In other embodiments, the invention provides a method comprising detecting fluorescence emitted from the fluorochrome conjugate and constructing an image. With respect to in vivo imaging, the method comprises (a) administering to a subject fluorochrome conjugate of the invention to a subject; (b) allowing the fluorochrome conjugate to distribute within the subject; (c) illuminating the subject to light of a wavelength absorbable by the fluorochromes of the fluorochrome conjugate; and (d) detecting an optical signal emitted by the fluorochrome. The signal emitted by the fluorochrome can be used to construct an image, either alone or as fused (combined or composite) images with other imaging modalities, including but not limited to magnetic resonance, ultrasound, X-ray, and computed tomography images. In one embodiment, one or more of the images are a tomographic image. Furthermore, it is understood that the foregoing steps can be repeated at predetermined intervals thereby permitting evaluation of the subject over time.

In another aspect, the invention provides a method of treating a subject having a disorder such as a disorder characterized by unwanted cellular proliferation, the method comprising: (a) administering a fluorochrome conjugate of the invention to a subject; (b) allowing the fluorochrome conjugate to distribute within the subject (c) illuminating the fluorochrome conjugate with light of a wavelength sufficient to produce cytotoxic singlet oxygen. In certain embodiments, steps (a) through (c) are repeated over time. In specific embodiments, steps (a) through (c) are repeated once, twice or three times over time. In preferred embodiments, steps (a) through (c) are repeated as determined to achieve the desired objective and based on other factors known to those of skill in the art. In certain embodiments, steps (a) through (c) further include the step of activating the fluorescence conjugate within the subject prior to step (c).

In certain embodiments, the subject may be a vertebrate, for example, a mammal, for example, a human.

The in vivo imaging and treatment methods described above can be used to determine the presence, absence, of a disease and/or treat a disease in the subject. Exemplary diseases include, without limitation, autoimmune disease, bone disease, cancer, cardiovascular disease, environmental disease, dermatological disease, immunologic disease, inherited disease, infectious disease, metabolic disease, neurodegenerative disease, ophthalmic disease, and respiratory disease.

In certain embodiments, the disorder is a cancer, tumor, neoplasm, vascularization, cardiovascular disease, intravasation, extravasation, metastasis, arthritis, infection, Alzheimer's Disease, blood clot, atherosclerosis, melanoma, or osteosarcoma. In a further embodiment, the disorder is cancer. In addition, in vivo imaging method can be used to assess the effect of a compound or therapy by using the fluorochrome conjugates, wherein the subject is imaged prior to and after treatment with the compound or therapy, and the corresponding images are compared.

In another aspect, the invention provides a kit for treating a subject having a disorder characterized by unwanted cellular proliferation comprising one or more unit dosage forms of one or more fluorochrome conjugates the invention and instructions for use.

In certain embodiments, the kit further comprises one or more pharmaceutically acceptable vehicles.

In still other embodiments, the kit further comprising one or more devices that facilitate the illumination of a fluorocrhome conjugate.

### BRIEF DESCRIPTION OF THE DRAWINGS

FIG. 1 shows the design of a PDT conjugate with a molecular switch on a multiple antigenic peptide (MAP) core.

FIG. 2 shows the solid-phase synthesis and molecular structures of MAP-based fluorescent probes; conditions (i) 20% piperidine in DMF, Fmoc-Amino acid-OH, HBTU/HOBt/DIEPA in DMF, (ii) Fmoc-15-amino-4,7,10,13-tetraoxapentadecanoic acid/PyBOP/DIEPA in 9:1 NMP/DCM, 20% piperidine in DMF, (iii) CyTE-777, DCC/HOBt in anhydrous DMF, TFA/TIS.

FIG. 3 HPLC traces of the purified samples of fluorophore probes CyPEG-1, CyPEG-2, and CyPEG-3.

FIG. 4 depicts absorption spectra showing the effect of pegylation on dye aggregation of the probes. The aggregation of dyes was seen as a strong absorption at around 705 nm.

FIG. 5 depicts fluorescence spectra of ˜3 μM solutions (20% DMSO in 10 mM phosphate buffer, pH 7.4) of the probes compared with an equimolar solution of CyTE-777; excitation wavelength 750 nm. The insert is the expanded region of fluorescence spectra for CyPEG 1-3.

FIG. 6 shows the results of mechanistic studies, which show a strong aggregation peak (705 nm) observed in 20% DMSO almost disappeared in 99% DMSO.

FIG. 7(A) shows the effect of pegylation on the kinetic profile of the probe activation with cathepsin S in 20% DMSO solution of pH 7.4, as compared to the controls (no enzyme added) CyPEG-1C, CyPEG-2C, and CyPEG-3C; and FIG. 7(B) shows probe activation after 8 h at 810 nm as compared to the controls.

FIG. 8 shows CyPEG-2 activation and selectivity under optimized pH conditions; cathepsin S: pH 6.5, 10 mM phosphate buffer with 20% DMSO; cathepsin S/E-64: pH 6.5, 10 mM phosphate buffer with 20% DMSO in the presence of E-64 protease inhibitor, cathepsin L: pH 5.5, 10 mM phosphate buffer with 20% DMSO; cathepsin K: pH 4.5, 10 mM phosphate buffer with 20% DMSO; cathepsin S*: pH 6.5, 10 mM phosphate buffer.

### DETAILED DESCRIPTION

### Definitions

In order that the invention may be more readily understood, certain terms are first defined and collected here for convenience. Other definitions appear in context throughout the application.

The term “antibody” as used in this invention includes intact immunoglobulin molecules as well as fragments thereof, such as Fab and Fab′, which are capable of binding an epitopic determinant. Fab fragments retain an entire light chain, as well as one-half of a heavy chain, with both chains covalently linked by the carboxy terminal disulfide bond.

As used herein, “backbone” means a biocompatible moiety to which fluorochromes are covalently linked in fluorescence-quenching positions.

The term “cancer” refers to a malignant tumor of potentially unlimited growth that expands locally by invasion and systemically by metastasis. The term “cancer” also refers to the uncontrolled growth of abnormal cells.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “dendrimer” refers to a synthetic, multi-dimensional macromolecule with branching parts, including, but not limited to polymers having a regular branched structure and those built up from a monomer, with new branches added in steps until a tree-like structure is created.

As used herein, “fluorochrome attachment moiety” means a molecule to which two or more fluorochromes are covalently linked (directly or through a spacer) and maintained in fluorescence-quenching positions relative to one another.

A “fluorochrome” is a molecule that becomes fluorescent or self-luminous after exposure to light including, but not limited to, a fluorochrome, a fluorophore, a fluorochrome quencher molecule, or any fluorescent organic or inorganic molecule that becomes fluorescent or self-luminous after exposure to light.

As used herein, “quenching positions” means the interaction-permissive positions of two or more atoms (in a single polymer) to which fluorochromes can be covalently linked (directly or indirectly through a spacer) so that the fluorochromes are maintained in a position relative to each other that permits them to interact photochemically and quench each other's fluorescence and/or singlet oxygen generation.

A “peptide” is a sequence of at least two amino acids. Peptides can consist of short as well as long amino acid sequences, including full length proteins.

As used herein, “photoactivation” means a light-induced chemical reaction of a photosensitizer, which produces a biological effect.

The term “photosensitizer” refers to a photoactivatable compound, or a biological precursor thereof, that produces a reactive species (e.g., oxygen) having a photochemical (e.g., cross linking) or phototoxic effect on a cell, cellular component or biomolecule. A “photosensitizer” is a type of “fluorochrome.”

A “protease cleavage site” is an amino acid sequence that serves as a cleavable substrate for proteolytic enzymes.

A “solubility enhancing agent” is a moiety linked to a compound that enhances the solubility of the compound in a solvent.

A “spacer” is an atom, group of atoms, compound or moiety used to facilitate interaction of a peptide substrate with the active site of the enzyme.

The term “subject” refers to animals such as mammals, including, but not limited to, primates (e.g., humans), cows, sheep, goats, horses, dogs, cats, rabbits, rats, mice and the like. In certain embodiments, the subject is a human.

As used herein, “targeting moiety” means a moiety bound covalently or noncovalently to a conjugate which moiety enhances the concentration of the conjugate in a target tissue relative to surrounding tissue.

“Unwanted cellular proliferation” refers to hyperproliferative and/or neoplastic cells, and include those cells having the capacity for unregulated or abnormally regulated growth.

It is also to be understood that the terminology used herein is for purposes of describing particular embodiments only, and is not intended to be limiting. As used in the specification and the appended claims, the singular forms “a”, “an”, and “the” include plural referents unless the context clearly indicates otherwise. Thus, for example, reference to “a peptide” includes multiple peptides, reference to “a spacer” includes two or more spacers.

Unless otherwise defined, all technical and scientific terms used herein have the same meaning as commonly understood by one of ordinary skill in the art to which this invention belongs. In case of conflict, the present application, including definitions will control. All publications, patent applications, patents, and other references mentioned herein are incorporated by reference.

### Proteases and Disease Progression

Proteases are enzymes that catalyze hydrolysis of peptide amide bonds. Changes in the regulation of protease are a common feature of many diseases, such as neoplastic, vascular, infectious, degenerative and autoimmune disorders (Table 1).

TABLE 1
Examples of key proteases and diseases

| Diseases | Proteases |
|---|---|
| Cancer growth | Cathepsins |
| Metastasis | Matrix Metalloproteinases |
| Apoptosis | Caspases |
| Arthritis | Matrix Metalloproteinases |
| Infection | Viral proteases |
| Alzheimer | Secretases |
| Blood clotting | Thrombin |
| Atherosclerosis | Matrix Metalloproteinases, |
| Cardiovascular | Cathepsins |
| Disease | |
| Osteoporosis | Cathepsin K |

Tumor progression is a complex, multi-stage process by which a normal cell undergoes genetic changes that result in phenotypic alterations and the acquisition of the ability to spread and colonize distant sites in the body. Proteases are known to function at multiple stages of tumor progression, affecting tumor establishment, growth, neovascularization, intravasation, extravasasion and metastasis.

Among the many tumor-associated proteases, matrix metalloproteinases (MMPs) are prime candidates for mediating tumor progression because MMP-family members collectively degrade all structural components of the extracellular matrix (ECM). There exist at least four distinct subsets including collagenases, gelatinases, stromelysins and membrane-type MMPs while at least 25 protein members have been recognized in total (Table 2). MMPs, a family of enzymes highly homologous to zinc endopeptidases, degrade collagens, gelatins, fibronectin, and laminin. Both normal and transformed cells can produce one or more members of the MMP family. Numerous studies have shown a close association between MMP expression and proliferation, invasive behavior and metastatic potential of tumors. MMPs also play important roles in normal connective tissue turnover during morphogenesis, development, wound healing, reproduction, and neovascularization. In addition to MMPs, other proteases, such as cathepsin B, cathepsin D, prostate specific antigen, and plasminogen activator, have been found to be involved in the development of various cancers.

TABLE 2
Enzymes of the MMP family

| | | MMP | |
|---|---|---|---|
| Subclass | Enzyme | number | Main Substrates |
| Gelatinases | Gelatinase A | MMP-2 | Type IV, V and fibrillar |
| | | | collagens, Fibronectin |
| | Gelatinase B | MMP-9 | Type IV and V collagens |
| Collagenases | Interstitial | MMP-1 | Fibrillar collagens |
| | collagenase | | |
| | Neutrophil | MMP-8 | Fibrillar collagens |
| | collagenase | | |
| | Collagenas-3 | MMP-13 | Fibrillar collagens |
| Stromelysins | Stromelysin-1 | MMP-3 | Non-fibrillar collagen, |
| | | | laminin, fibronectin, |
| | Stromelysin-2 | MMP-10 | Non-fibrillar collagen, |
| | | | laminin, fibronectin, |
| | Stromelysin-3 | MMP-11 | Non-fibrillar collagen, |
| | | | laminin, fibronectin, |
| | Matrilysin | MMP-7 | Non-fibrillar collagen, |
| | | | laminin, fibronectin, |
| Membrane | MT1-MMP | MMP-14 | ProMMP-2, gelatin, |
| types | | | collagens |
| | MT2-MMP | MMP-15 | ProMMP-2 |
| | MT3-MMP | MMP-16 | ProMMP-2 |
| | MT4-MMP | MMP-17 | |
| | MT5-MMP | MMP-21 | |
| Others | Metalloelastase | MMP-12 | Elastin |
| | RASI-1 | MMP-18/19 | |
| | Enamelysin | MMP-20 | Amelogenin |

### Protease Cleavage and Cleavage Sites

In certain embodiments, the protease cleavage site is cleaved by proteases including but not limited to cathepsins, matrix metalloproteinases (MMP), membrane-type MMPs, collagenases, gelatinases, stromelysins, caspases, viral proteases, HIV proteases, HSV proteases, gelatinase, urokinases, secretases, endosomal hydrolase, Prostate Specific Antigen (PSA), plasminogen activator, Cytomegalovirus (CMV) protease, and thrombin. In further embodiments, the cathepsin is Cathepsin, Cathepsin B, Cathepsin D, Cathepsin H, Cathepsin K, Cathepsin L, or Cathepsin S. Some conjugates naturally accumulate in proliferative cells, for example, comprising tumor interstitium or tumor cells, e.g., by fluid phase endocytosis. By virtue of this preferential accumulation, such conjugates can be used to image and treat tumor tissues, even if the protease(s) activating the conjugate is not tumor specific.

In specific embodiments, the protease cleavage site is an amino acid sequence including but not limited to, RR, RRG, GPICFFRLG (SEQ. ID. NO. 1), HSSKLQG (SEQ: ID. NO. 2), PIC(Et)FF (SEQ. ID. NO. 3), HSSKLQ (SEQ. ID. NO. 4), P(L/Q)G(I/L)AG (SEQ. ID. NO. 5), GVVQASCRLA (SEQ. ID. NO. 6) or KK. Preferably, the sequence is Leu-Arg.

### Design of Conjugates

Guidance concerning various components of the conjugates, including backbone, protective side chains, fluorochromes, photosensitizers, photosensitizer attachment moieties, spacers, cleavage sites and targeting moieties is provided in the paragraphs below.

Conjugates of the invention comprise a backbone. The backbone design will depend on considerations such as biocompatibility (e.g., toxicity and immunogenicity), serum half-life, useful functional groups (for conjugating fluorochromes, spacers, and protective groups), and cost. Useful types of backbones include polypeptides (polyamino acids), nucleic acids, synthetic polymers, polyethyleneamines, polysaccharides, aminated polysaccharides, aminated oligosaccharides, polyamidoamines, polyacrylic acids, and polyalcohols, including dendrimers of the aformentioned. In some embodiments, the backbone consists of a polypeptide formed from L-amino acids, D-amino acids, or a combination thereof. Such a polypeptide can be, e.g., a polypeptide identical or similar to a naturally occurring protein such as albumin, a homopolymer such as poly-L-ysine, or a copolymer such as a D-tyr-D-lys copolymer. In certain embodiments, the branched polypeptide is poly-L-lysine. In certain embodiments, the backbone comprises albumin, antibodies, or antibody fragments. In certain embodiments, the backbone is a synthetic polymer wherein said polymer is polyglycolic acid, polylactic acid, poly(glycolic-colactic) acid, polydioxanone, polyvalero lactone, poly-ε-caprolactone, poly(3-hydroxybutyrate), poly(3-hydroxyvalerate) polytartronic acid, or poly(β-malonic acid).

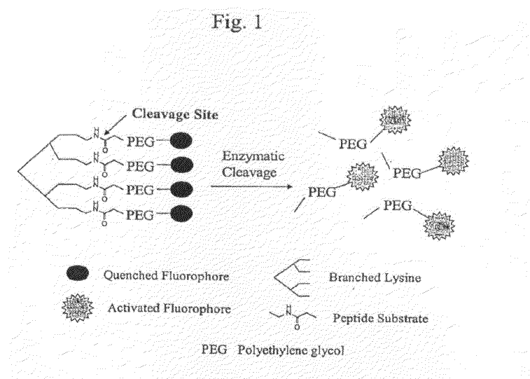

It is particularly desirable to make discrete peptide-based dendrimers that are smaller, thus facilitating their rapid clearance and precise characterization. The multiple antigenic peptide (MAP) system (Tam, J. P. Proc. Natl. Acad. Sci. USA 1988, 85, 5409-5413; Crespo, L.; Sanclimens, G.; Pons, M.; Giralt, E.; Royo, M.; Albericio, F. Chem. Rev. 2005, 105, 1663-1681) is a small and discrete, dendrimeric scaffold. It has been observed that peptides making the dendritic arms of the MAP system have a tendency to aggregate (Tam, J. P. Proc. Natl. Acad. Sci. USA 1988, 85, 5409-5413). Therefore, fluorochromes (e.g., photosensitizers) can be quenched by aggregation if they are attached to the termini of the dendritic arms of the MAP system. The quenched conjugate can be targeted by proteolytic enzymes, for example, through incorporation of a corresponding protease cleavage site in the dendritic arms. The conjugate will then fluoresce only after activation by the enzymatic cleavage of the peptide bond (FIG. 1).

In some embodiments, the dendritic backbone contains a small number of amino acids, e.g., 5 to 20 amino acids, with photosensitizers attached to amino acids on opposite sides of a protease cleavage site. In some embodiments, the dendritic backbone contains 5 to 15 amino acids, more preferably 5, 6, 7, 8, 9, 10, 11, 12, 13, 14, or 15 amino acids.

In some embodiments the dendrimers include STARBURST PAMAM (polyamidoamine) dendrimers, dense star dendrimers, non-dense star dendrimers, arborol dendrimers, self-immolative dendrimers, polypropyleneimine (PPI) dendrimers, phosphorous containing dendrimers, and other different types of dendrimers (Polyester, POPAM, porphyrin-, podand-, organometallic-, silicon-based) with various cascade architectures.

The conjugate can comprise one or more spacer molecules, preferrably one, two, three, four or more spacer molecules. In some embodiments of the invention, the fluorochromes are linked directly or indirectly to the dendrimeric backbone through spacers. In certain embodiments, a spacer molecule is a peptide, oligopeptide, polysaccharide, a nucleic acid, or a synthetic cleavable moiety. In a further embodiment, a spacer molecule is a peptide, such as glycine, β-alanine, and other natural or unnatural amino acids.

In some embodiments of the invention, fluorochromes and/or photosensitizers are linked to the backbone through peptides containing protease cleavage sites. In other embodiments, the protease cleavage sites are located within the backbone, for example, where the backbone comprises a polypeptide-based dendrimer. Peptide spacers can be designed to contain amino acid sequences recognized by specific proteases associated with target tissues.

In some embodiments of the invention, paired fluorochromes and/or photosensitizers in fluorescence-quenching positions are in a single polypeptide side chain containing a protease cleavage site between the fluorochromes. Such a side chain can be synthesized as an activatable fluorescence module that can be used as a probe per se, or covalently attached to a backbone (carrier) or targeting molecule, e.g., an albumin, antibody, receptor binding molecule, synthetic polymer or polysaccharide. A useful conjugation strategy is to place a cysteine residue at the N-terminus or C-terminus of the module and then employ SPDP for covalent linkage between the side chain of the terminal cysteine residue and a free amino group of the carrier or targeting molecule.

When the fluorochromes/photosensitizers are linked directly to the backbone, activation occurs by cleavage site located within the backbone. High fluorochrome loading of the backbone can potentially interfere with backbone cleavage by activating enzymes such as trypsin. Therefore, a balance between fluorescence quenching and accessibility of the backbone by activating enzymes is needed, and well within the skill of one in the art to formulate. For any given backbone-fluorochrome combination (when activation sites are in the backbone) a range of fluorochrome loading densities can be produced and tested in vitro to determine the optimal fluorochrome loading percentage.

When the fluorochromes and/or photosensitizers are linked to the backbone through spacers containing protease cleavage sites, accessibility to the backbone by proteases is unnecessary. Therefore, high loading of the backbone with spacers and fluorochromes does not significantly interfere with activation. In such a system, for example, every lysine residue of polylysine can carry a spacer and fluorochrome, and every fluorochrome can be released by protease cleavage.

The conjugate can also comprise at least one solubility enhancing group. In certain embodiments, the solubility is increased in aqueous solutions. Examples of solubility enhancing groups include polyethylene glycol (PEG), methoxypolyethylene glycol (MPEG), methoxypolypropylene glycol, copolymers of polyethylene glycol and methoxypolypropylene glycol, dextran, and polylactic-polyglycolic acid, polyethylene glycol-diacid, PEG monoamine, MPEG monoamine, MPEG hydrazide, MPEG imidazole, copolymers of polyethylene glycol, methoxypolypropylene glycol, polyethylene glycol-diacid, polyethylene glycol monoamine, MPEG monoamine, MPEG hydrazide, MPEG imidazole or mixtures thereof. The solubility enhancing groups can also be a block-copolymer of PEG and a different polymer such as a polypeptide, polysaccharide, polyamidoamine, polyethyleneamine or polynucleotide. PEG-1 refers to a one-unit PEG group. PEG-2 refers to a two-unit PEG group, and PEG-3 refers to a three-unit PEG group. Synthetic, biocompatible polymers are discussed generally in Holland et al., 1992, “Biodegradable Polymers,” Advances in Pharmaceutical Sciences 6:101-164. In certain embodiments, the solubility enhancing group is PEG. A useful backbone-protective chain combination is methoxypoly(ethylene)glycol-succinyl-N-.epsilon.-poly-L-lysine (PL-MPEG). The synthesis of this material, and other polylysine backbones with protective chains, is described in Bogdanov et al., U.S. Pat. No. 5,593,658 and Bogdanov et al., 1995, Advanced Drug Delivery Reviews 16:335-348.

The solubility enhancing agent is typically inserted between the fluorochrome and the peptide substrate. In certain embodiments, the solubility enhancing agent is attached to the peptide substrate via a spacer molecule.

A photosensitizer and a solubility enhancing agent together form a photosensitive moiety. In certain embodiments, the solubility enhancing groups include polyethylene glycol (PEG), methoxypolyethylene glycol (MPEG), methoxypolypropylene glycol, copolymers of polyethylene glycol and methoxypolypropylene glycol, dextran, and polylactic-polyglycolic acid, polyethylene glycol-diacid, PEG monoamine, MPEG monoamine, MPEG hydrazide, MPEG imidazole, copolymers of polyethylene glycol, methoxypolypropylene glycol, polyethylene glycol-diacid, polyethylene glycol monoamine, MPEG monoamine, MPEG hydrazide, MPEG imidazole or mixtures thereof.

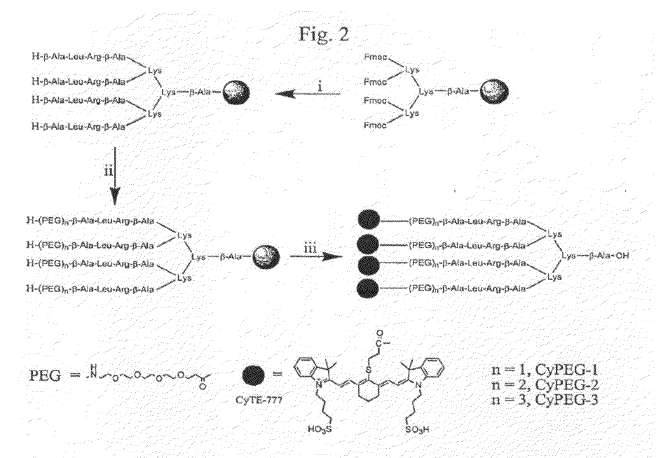

The synthetic scheme and molecular structures of exemplary conjugates are given in FIG. 2. The conjugates are based on a tetravalent, branched lysine core. Extending from the core are the dendritic arms that incorporate a dipeptide, Leu-Arg, as a substrate (Bromme, D., et al. J. Biol. Chem. 1996, 271, 2126-2132; Bossard, M. J. et al. J Biol. Chem. 1996, 271, 12517-12524) for the targeted protease, cathepsin S (“cathepsin S conjugate”). In order to facilitate the interaction of the peptide substrate with the active site of the enzyme, β-alanine is attached as a spacer molecule on either side of the peptide substrate. To the four N-termini of this peptidic scaffold are attached CyTE-777, a near-infrared fluorescent dye. The fluorochrome comprises a central carboxylic acid for conjugation to biomolecules, has absorption (777 nm) and emission (812 nm) bands similar to indocyanine green, and shows no tendency to aggregate in aqueous media.

Because of the branched lysine core, however, aggregation of the dye molecules within the conjugate occurs. While dye aggregation provides efficient quenching, it can result in decreased aqueous solubility. To optimize the aqueous solubility of the conjugates, short and discrete polyethylene glycol (PEG) moieties are inserted between the fluorophores and the peptides. PEG has a dynamic conformation and is well hydrated in aqueous media, which results in improved aqueous solubility of a pegylated molecule (Caliceti, P. et al. Adv. Drug Deliv. Rev. 2003, 55, 1261-1277). PEG can be incorporated in the dendritic arms to achieve balance between the dye aggregation and the aqueous solubility of the conjugate.

A preferred non-toxic photosensitizer conjugate can be constructed with a dendritic core, multiple protease linkers, multiple porphyrin or chlorin-based photosensitizers, and multiple short PEG chains. The dendritic core will advantageously constrain the photosensitizer, the peptide spacer can facilitate protease selectivity, and the PEG chains enhance aqueous solubility. The pro-photosensitizer is optically silent due to intramolecular quenching. Intramolecular quenching limits intersystem crossing from the singlet excited state to the triplet excited state which is essential to generate cytotoxic singlet oxygen.

A multiple antigen peptide (“MAP”)-based system can be utilized for making a conjugate in which the photosensitizer has maximum quenching efficiency. This design has two important features: 1) multivalency for effective self-quenching, and 2) the ability to promote aggregation of the photosensitizers that are otherwise in a free state in aqueous solutions. The core of the MAP system originates from a single amino acid residue and then extends into a branched structure in one direction where the photosensitizer will be attached to the peptide substrate or PEG chain. Another advantage of a MAP core is its flexibility, different number of branching arms can be conveniently synthesized using the solid phase synthesis according to methods known in the art.

### Fluorochromes and Photosensitizers

Fluorochromes of the present invention can be be any known in the art, including, but not limited to 6-carboxy-4′,5′-dichloro-2′,7′-dimethoxyfluorescein succinimidyl ester; 5-(and-6)-carboxyeosin; 5-carboxyfluorescein; 6-carboxyfluorescein; 5-(and-6)-carboxyfluorescein; 5-carboxyfluorescein-bis-(5-carboxymethoxy-2-nitrobenzyl)ether, -alanine-carboxamide, or succinimidyl ester; 5-carboxyfluorescein succinimidyl ester; 6-carboxyfluorescein succinimidyl ester, 5-(and-6)-carboxyfluorescein succinimidyl ester; 5-(4,6-dichlorotriazinyl) aminofluorescein; 2′,7′-difluorofluorescein; eosin-5-isothiocyanate; erythrosin-5-isothiocyanate; 6-(fluorescein-5-carboxamido) hexanoic acid or succinimidyl ester; 6-(fluorescein-5-(and-6)-carboxamido) hexanoic acid or succinimidyl ester; fluorescein-5-EX succinimidyl ester; fluorescein-5-isothiocyanate; fluorescein-6-isothiocyanate; Oregon Green® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidyl ester or triethylammonium salt; Oregon Green® 514 carboxylic acid; Oregon Green® 514 carboxylic acid or succinimidyl ester; Rhodamine Green™ carboxylic acid, succinimidyl ester or hydrochloride; Rhodamine Green™ carboxylic acid, trifluoroacetamide or succinimidyl ester; Rhodamine Green™-X succinimidyl ester or hydrochloride; Rhodol Green™ carboxylic acid, N,O-bis-(trifluoroacetyl) or succinimidyl ester; bis-(4-carboxypiperidinyl) sulfonerhodamine or di(succinimidyl, ester); 5-(and-6)-carboxynaphthofluorescein, 5-(and-6)-carboxynaphthofluorescein succinimidyl ester; 5-carboxyrhodamine 6G hydrochloride; 6-carboxyrhodamine 6G hydrochloride, 5-carboxyrhodamine 6G succinimidyl ester; 6-carboxyrhodamine 6G succinimidyl ester; 5-(and-6)-carboxyrhodamine 6G succinimidyl ester; 5-carboxy-2′,4′,5′,7′-tetrabromosulfonefluorescein succinimidyl ester or bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodamine succinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green isothiocyanate; NANOGOLD® mono(sulfosuccinimidyl ester); QSY® 21 carboxylic acid or succinimidyl ester; QSY® 7 carboxylic acid or succinimidyl ester; Rhodamine Red™-X succinimidyl ester; 6-(tetramethylrhodamine-5-(and-6)-carboxamido)hexanoic acid succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; and X-rhodamine-5-(and-6)-isothiocyanate.

Fluorescent dyes of the present invention can be, for example, BODIPY® dyes commercially available from Molecular Probes, including, but not limited to BODIPY® FL; BODIPY® TMR STP ester; BODIPY® TR-X STP ester; BODIPY® 630/650-X STP ester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoic acid; 4,4-difluoro-5,7-dimethyl-4-bora-3a,4a-diaza-s-indacene-3-pentanoic acid succinimidyl ester; 4,4-difluoro-5,7-dimethyl-4-bora-3a,4a-diaza-s-indacene-3-propionic acid; 4,4-difluoro-5,7-dimethyl-4-bora-3a,4a-diaza-s-indacene-3-propionic acid succinimidyl ester; 4,4-difluoro-5,7-dimethyl-4-bora-3a,4a-diaza-s-indacene-3-propionic acid sulfosuccinimidyl ester or sodium salt; 6-((4,4-difluoro-5,7-dimethyl-4-bora-3a,4a-diaza-s-indacene-3-propionyl)amino)hexanoic acid; 6-((4,4-difluoro-5,7-dimethyl-4-bora-3a,4a-diaza-s-indacene-3-propionyl)amino)hexanoic acid or succinimidyl ester; N-(4,4-difluoro-5,7-dimethyl-4-bora-3a,4a-diaza-s-indacene-3-propionyl)cysteic acid, succinimidyl ester or triethylammonium salt; 6-4,4-difluoro-1,3-dimethyl-5-(4-methoxyphenyl)-4-bora-3a,4a 4,4-difluoro-5,7-diphenyl-4-bora-3a,4a-diaza-s-indacene-3-propionic acid; 4,4-difluoro-5,7-diphenyl-4-bora-3a,4a-diaza-s-indacene-3-propionic acid succinimidyl ester; 4,4-difluoro-5-phenyl-4-bora-3a,4a-diaza-s-indacene-3-propionic acid succinimidyl ester; 6-((4,4-difluoro-5-phenyl-4-bora-3a,4a-diaza-s-indacene-3-propionyl)amino)hexanoic acid or succinimidyl ester; 4,4-difluoro-5-(4-phenyl-1,3-butadienyl)-4-bora-3a,4a-diaza-s-indacene-3-propionic acid succinimidyl ester; 4,4-difluoro-5-(2-pyrrolyl)-4-bora-3a,4a-diaza-s-indacene-3-propionic acid succinimidyl ester, 6-(((4,4-difluoro-5-(2-pyrrolyl)-4-bora-3a,4a-diaza-s-indacene-3-yl)styryloxy)acetyl)aminohexanoic acid or succinimidyl ester; 4,4-difluoro-5-styryl-4-bora-3a,4a-diaza-s-indacene-3-propionic acid; 4,4-difluoro-5-styryl-4-bora-3a,4a-diaza-s-indacene-3-propionic acid succinimidyl ester; 4,4-difluoro-1,3,5,7-tetramethyl-4-bora-3a,4a-diaza-s-indacene-8-propionic acid; 4,4-difluoro-1,3,5,7-tetramethyl-4-bora-3a,4a-diaza-s-indacene-8-propionic acid succinimidyl ester; 4,4-difluoro-5-(2-thienyl)-4-bora-3a,4a-diaza-s-indacene-3-propionic acid succinimidyl ester; 6-(((4-(4,4-difluoro-5-(2-thienyl)-4-bora-3a,4a-diaza s-indacene-3-yl)phenoxy)acetyl) amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester.

Fluorescent dyes the present invention can be, for example, Alexa fluor dyes commercially available from Molecular Probes, including but not limited to Alexa Fluor® 350 carboxylic acid; Alexa Fluor® 430 carboxylic acid; Alexa Fluor® 488 carboxylic acid; Alexa Fluor® 532 carboxylic acid; Alexa Fluor® 546 carboxylic acid; Alexa Fluor® 555 carboxylic acid; Alexa Fluor® 568 carboxylic acid; Alexa Fluor® 594 carboxylic acid; Alexa Fluor® 633 carboxylic acid; Alexa Fluor® 647 carboxylic acid; Alexa Fluor® 660 carboxylic acid; and Alexa Fluor® 680 carboxylic acid. Fluorescent dyes the present invention can also be, for example, cyanine dyes commercially available from Amersham-Pharmacia Biotech, including, but not limited to Cy3 NHS ester; Cy 5 NHS ester; Cy5.5 NHS ester; and Cy 7 NHS ester or fluorophores commercially available from VisEn Medical, Inc, (Woburn, Mass.) such as VivoTag680 and VivoTag750.

Photosensitizers known in the art are typically selected for use according to: 1) efficacy in delivery, 2) proper localization in target tissues, 3) wavelengths of absorbance, 4) proper excitatory wavelength, 5) purity, 6) quenching property, including fluorescence and singlet oxygen generation, and 7) in vivo effects on pharmacokinetics, metabolism, and maximum phototoxicity.

A photosensitizers for clinical use is optimally amphiphilic, meaning that it shares the opposing properties of being water-soluble, yet hydrophobic. The photosensitizer should be water-soluble in order to pass through the bloodstream systemically, however it should also be hydrophobic enough to pass across cell membranes. Modifications, such as attaching polar residues (amino acids, sugars, and nucleosides) to the hydrophobic porphyrin ring, can alter polarity and partition coefficients to desired levels. Such methods of modification are well known in the art.

In some embodiments, photosensitizers absorb light at a relatively long wavelength, thereby absorbing at low energy. Low-energy light can travel further through tissue than high-energy light, which becomes scattered. Optimal tissue penetration by light occurs between about 650 and about 800 nm. Porphyrins found in red blood cells typically absorb at about 630 nm, and new, modified porphyrins have optical spectra that have been “red-shifted”, in other words, absorbs lower energy light. Other naturally occurring compounds have optical spectra that is red-shifted with respect to porphyrin, such as chlorins found in chlorophyll (about 640 to about 670 nm) or bacteriochlorins found in photosynthetic bacteria (about 750 to about 820 nm).

Photosensitizers of the invention can be any known in the art, and optionally coupled to molecular carriers. For example, porphyrins and hydroporphyrins can include, but are not limited to, Photoftin® (porfimer sodium), hematoporphyrin LX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylene dicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorin e₆ monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium(M) texaphyrin.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at about 630 nm and have an overall low fluorescent quantum yield and low efficiency in generating reactive oxygen species. Light at about 630 nm can only penetrate tissues to a depth of about 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generation porphyrins.

Chlorins and bacteriochlorins are also porphyrin derivatives; however these have the unique property of hydrogenated exo-pyrrole double bonds on the porphyrin ring backbone, allowing for absorption at wavelengths greater than about 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further Ted-shifted to about 740 nm. A specific embodiment of the invention uses chlorinₑ₆.

Purpurins, porphycenes, and verdins are also porphyrin derivatives that have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals. In a specific embodiment, the photosensitizer can be Antrin®, otherwise known as motexafin lutetium.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX, is dependent on the rate of 5-aminolevulinic acid synthesis, governed in a negative-feedback manner by concentration of free heme. Conversion of protoporphyrin IX is slow, and where desired, administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a specific embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized (Richter et al., 1987; Aveline et al., 1994; Levy, 1994) and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically administered intravenously, with an optimal incubation time range from 1.5 to 6 hours. Verteporfin absorbs at 690 nm, and is activated with commonly available light sources. One tetrapyrrole-based photosensitizer having recent success in the clinic is MV0633 (Miravant). MV0633 is well suited for cardiovascular therapies and as such, can be used in therapeutic and diagnostic methods of the invention.

In specific embodiments, the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength. Such specific embodiments include motexafin lutetium (Antrin®) and chlorinₑ₆.

Photosensitizers of the invention can be any known in the art, including photofrin, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hem atoporphyrin, ALA-induced protoporphyrin IX, endogenous metabolic precursors, 5-aminolevulinic acid benzonaphthoporphyrazines, cationic imminium salts, tetracyclines, lutetium texaphyrin, tin-etio-purpurin, porphycenes, benzophenothiazinium and combinations thereof.

Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maxima at about 680 nm. Phthalocyanines, belonging to a new generation of substances for PDT are chelated with a variety of diamagnetic metals, chiefly aluminum and zinc, which enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, where, for example, high tumor to tissue ratios (8:1) were observed after 1-3 hours. The cyanines are eliminated rapidly and almost no fluorescence can be seen in the tissue of interest after 24 hours.

Cyanine and other dyes include but are not limited to merocyanines, phthalocyanines with or without metal substituents, chloroaluminum phthalocyanine with or without varying substituents, sulfonated aluminum PC, ring-substituted cationic PC, sulfonated AlPc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds and phenothiazine derivatives including methylene blue.

Other photoactive dyes such as methylene blue and rose bengal, are also used for photodynamic therapy. Methylene blue is a phenothiazine cationic dye that is exemplified by its ability to specifically target mitochondrial membrane potential. Rose-bengal and fluorescein are xanthene dyes that are well documented in the art for use in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, and tetracyclines are other compounds that also exhibit photoactive properties and can be used advantageously in photodynamic therapy. Other photosensitizers that do not fall in either of the aforementioned categories have other uses besides photodynamic therapy, but are also photoactive. For example, anthracenediones, anthrapyrazoles, aminoanthraquinone compounds are often used as anticancer therapies (i.e. mitoxantrone, doxorubicin). Chalcogenapyrylium dyes such as cationic selena- and tellurapyrylium derivatives have also been found to exhibit photoactive properties in the range of about 600 to about 900 nm range, more preferably from about 775 to about 850 nm. In addition, antibiotics such as tetracyclines and fluoroquinolone compounds have demonstrated photoactive properties.

In an attempt to overcome those problems, a prophyrin compound which is a single compound and exhibits its adsorption in a longer wavelength region (650-800 nm) has been proposed as a second generation agent for PDT. Examples of such second generation agent includes amino-levulinic acid (ALA) which is a protoporphyrin precursor; asparthyl-chlorin e6 (Np e6) which is a chlorin derivative; benzoporphyrin derivative (BPD) and methatetrahydroxyphenylchlorin (m-THPC), both of which are new type of chlorin derivatives obtained by the structural conversion from hemoglobin-derived porphyrins.

In addition, the present inventors proposed chlorin derivatives and the analogues thereof, e.g., an alkoxyiminochlonyl aspartic acid derivative (Japanese Patent Application Laid-open Nos. 5-97857 and 9-124652), confirming that these compounds are useful as photosensitizers for PDT.

The fluocorchrome conjugates of the present invention can comprise any combination of fluorochromes and photosensitizers. In certain embodiments, fluorochrome conjugates can be constructed to comprise two or more distinct fluorochromes or photosensitizers conjugated to a single backbone such that one fluorochrome or photosensitizer absorbs and emits fluorescent light at a distinct wavelength from the other fluorochomes or photosensitizers. Such dual- or multi-component fluorochrome conjugates can be used for dual or multi-imaging and phototherapy purposes. For example, such dual or multi-component conjugates can be used to absorb and emit fluorescent light at one wavelength such that the cells targeted are illuminated to confirm accumulation in specific target tissue. The same dual or multi-component conjugates can then be used to absorb and emit fluorescent light at a distinct wavelength to produce singlet oxygen thereby killing or damaging the target cells. The activation sites attaching the fluorochromes and photosensitizer molecules to the backbone, may be the same or different.

### Excitations and Emissions

Fluorochrome conjugates with excitation and emission wavelengths in the near infrared spectrum are desirable, i.e., 500-1300 nm. Use of this portion of the electromagnetic spectrum maximizes tissue penetration and minimizes absorption by physiologically abundant absorbers such as hemoglobin (<650 nm) and water (>1200 nm). Ideal near infrared fluorochromes for in vivo use exhibit: (1) narrow spectral characteristics, (2) high sensitivity (quantum yield), (3) biocompatibility, and (4) decoupled absorption and excitation spectra.

Intramolecular quenching by non-activated fluorochromes can occur by any of various quenching mechanisms. Several mechanisms are known, including resonance energy transfer between two fluorochromes. In this mechanism, the emission spectrum of a first fluorochrome should be very similar to the excitation of a second fluorochrome, which is in close proximity to the first fluorochrome. Self-quenching can also result from fluorochrome aggregation or excimer formation. This effect is strictly concentration dependent. Quenching also can result from a non-polar-to-polar environmental change.

To achieve intramolecular quenching, several strategies can be applied. They include: (1) linking a second fluorochrome, as an energy acceptor, at a suitable distance from the first fluorochrome; (2) linking fluorochromes to the backbone at high density, to induce self-quenching; and (3) linking polar fluorochromes in a vicinity of non polar structural elements of the backbone and/or protective chains. Fluorescence is partially or fully recovered upon cleavage of the fluorochrome from neighboring fluorochromes and/or from a particular region, e.g., a non-polar region, of the probe.

Accumulation in Target Tissue

Preferential accumulation in a target tissue can be achieved or enhanced by binding a tissue-specific targeting moiety (e.g., targeting ligand) to the conjugate. The binding can be covalent or non-covalent. Examples of targeting moieties include a monoclonal antibody (or antigen-binding antibody fragment) directed against a target-specific marker, a receptor-binding polypeptide directed to a target-specific receptor, and a receptor-binding polysaccharide directed against a target-specific receptor. Antibodies or antibody fragments can be produced and conjugated to probes of this invention using conventional antibody technology (see, e.g., Folli et al., 1994, “Antibody-Indocyanin Conjugates for Immunophotodetection of Human Squamous Cell Carcinoma in Nude Mice,” Cancer Res. 54:2643-2649; Neri et al., 1997, “Targeting By Affinity-Matured Recombinant Antibody Fragments of an Angiogenesis Associated Fibronectin Isoform,” Nature Biotechnology 15:1271-1275). Similarly, receptor-binding polypeptides and receptor-binding polysaccharides can be produced and conjugated to probes of this invention using known techniques such as folate-mediated targeting (Leamon & Low, Drug Discovery Today, 6:44-51, 2001), transferrin, vitamins, carbohydrates and ligands that target internalizing receptors, including, but not limited to, asialoglycoprotein receptor, somatostatin, nerve growth factor, oxytocin, bombesin, calcitonin, arginine vasopressin, angiotensin II, atrial natriuretic peptide, insulin, glucagons, prolactin, gonadotropin, various opioids and urokinase-type plasminogen activator. Non-limiting examples include small molecules and peptide sequences to target integrins such as αᵥβ₃ and GPα_{IIb}β₃, bombesin, CD4 and VCAM-1). Also included are membrane, transmembrane, and nuclear translocation signal compounds and sequences, which can be derived from a number of sources including, without limitation, viruses and bacteria. Non-limiting examples include HIV-tat derived peptides, protamine, and polyArg and Arg-rich peptides. Importantly, targeting moeities can also include synthetic compounds including but not limited to small molecule drugs and derivatives thereof. Also included are antibiotics such as vancomycin, clindamycin, chemotherapeutics such as doxorubicin, molecules such as glycine, derivatives of AMG706, Zactima™, MP-412, erlotinib, sorafenib, dasatinib, lestaurtinib, lapatinib, XL647, XL999, MLN518, PKC412, STI571, AMN107, AEE788, OSI-930, OSI-817, sunitinib, AG-013736; molecules that target/inhibit VEGF receptors, PDGF receptor, HER2, SSKI, EphB4, EGFR, FGFR, VEGFR-2, VEGFR-3, serine/threonine and receptor kinases, FLT-3, type III RTKs, c-KIT, Bcr-Abl, CSF-1R, CCR-2, RET and VDGF-2.

### Devices and Methods for Imaging and Photoactivation

Typically, administration of fluorochrome conjugates is followed by a sufficient period of time to allow accumulation of the fluorochrome conjugate at the target site. Following this period of time, the fluorochrome conjugate is activated and can be irradiated with light for imaging and/or photoactivation. This is accomplished by applying light of a suitable wavelength and intensity, for an effective length of time, at the target site. As used herein, “irradiation” refers to the use of light to induced fluorescence to be emitted from a fluorochrome and/or a chemical reaction of a photosensitizer.

The suitable wavelength, or range of wavelengths, will depend on the particular fluorofluorochrome used, and can range from about 450 nm to about 950 nm. Particular suitable wavelengths include, but are not limited to wavelengths from about 450 nm to about 550 nm, from about 550 nm to about 650 nm, from about 650 nm to about 750 nm, from about 750 nm to about 850 nm and from about 850 nm to about 950 nm.

In specific embodiments, target tissues are illuminated with red light. Given that red and/or near infrared light best penetrates mammalian tissues, fluorochromes and/or photosensitizers with strong absorbances in the range of about 600 nm to about 900 nm are optimal for in vivo applications such as imaging and PDT. For irradiation, the wavelength of light is matched to the electronic absorption spectrum of the fluorochrome/photosensitizer so that the fluorochrome/photosensitizer absorbs photons and the desired photochemistry can occur. Wavelength specificity for irradiation generally depends on the molecular structure of the fluorochrome/photosensitizer. Photoactivation of photosentizers can also occur with sub-ablative light doses. Determination of suitable wavelength, light intensity, and duration of illumination is within ordinary skill in the art.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the fluorochrome/photosensitizer can alter these values. In general, fluorochromes/photosensitizers with longer absorbing wavelengths and higher molar absorption coefficients at these wavelengths are more effective imaging agents and photodynamic agents.

Photo activating dosages depend on various factors, including the amount of the photosensitizer administered, the wavelength of the photoactivating light, the intensity of the photoactivating light, and the duration of illumination by the photoactivating light. Thus, the dose can be adjusted to a therapeutically effective dose by adjusting one or more of these factors. Such adjustments are within the level of ordinary skill in the art.

The light for imaging and photoactivation can be produced and delivered to the target site by any suitable means known in the art. Light can be delivered to the target site from a light source, such as a laser or optical fiber. Preferably, optical fiber devices that directly illuminate the target site deliver the light. For example, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Other light delivery devices can be arthroscopes, laparascopes, brochoscopes, endoscopes, colonoscopies or hand held light delivery device. In addition, light can be transmitted by percutaneous instrumentation using optical fibers or cannulated waveguides. For open surgical sites, suitable light sources include broadband conventional light sources, broad arrays of light-emitting diodes (LEDs), and defocused laser beams.

Illumination can be by all methods known in the art, including transillumination. Some fluorochromes/photosensitizers can be illuminated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

Where treatment is desired, the dosage of photosensitizer composition, and light activating the photosensitizer composition, is administered in an amount sufficient to produce a phototoxic species. For example, where the photosensitizer is chlorine e6, administration to humans is in a dosage range of about 0.5 to about 10 mg/kg, preferably about 1 to about 5 mg/kg more preferably about 2 to about 4 mg/kg and the light delivery time is spaced in intervals of about 30 minutes to about 3 days, preferably about 12 hours to about 48 hours, and more preferably about 24 hours. The light dose administered is in the range of about 20-500 J/cm, preferably about 50 to about 300 J/cm and more preferably about 100 to about 200 J/cm. The fluence rate is in the range of about 20 to about 500 mw/cm, preferably about 50 to about 300 mw/cm and more preferably about 100 to about 200 mw/cm. Particular fluence rates are about 20 mw/cm, about 30 mw/cm, about 40 mw/cm, about 50 mw/cm, about 60 mw/cm, about 70 mw/cm, about 80 mw/cm, about 90 mw/cm, about 100 mw/cm, about 125 mw/cm, and about 150 mw/cm. There is a reciprocal relationship between photosensitizer compositions and light dose, thus, determination of suitable wavelength, light intensity, and duration of illumination is within ordinary skill in the art.

In performing methods of the invention, it is desirable for the phototoxic species to induce apoptosis and not necrosis of the cells comprising the vulnerable disorder. Lowering the fluence rate will favor apoptosis (e.g., less than about 100 mw/cm, e.g., about 10 to about 60 mw/cm, for chlorine e6). The wavelength and power of light can be adjusted according to standard methods known in the art to control the production of phototoxic species. Thus, under certain conditions (e.g., low power, low fluence rate, shorter wavelength of light or some combination thereof), a fluorescent species is primarily produced from the photosensitizer and any reactive species produced has a negligible effect. These conditions are easily adapted to bring about the production of a phototoxic species. For example, where the photosensitizer is chlorin e6, the light dose administered to produce a fluorescent species and an insubstantial reactive species is less than about 10 J/cm, preferably less than about 5 J/cm and more preferably less than about 1 J/cm. Determination of suitable wavelength, light intensity, and duration of illumination for any photosensitizer is within the level of ordinary skill in the art.

In a specific embodiment, photoactivation can be carried out using a specially designed intravascular device that delivers excitation light to the disorder and receives emitted fluorescence or other detectable signals (e.g., heat or radioactivity) that are transmitted to an analysis instrument. The same device can optionally be used to deliver therapeutic light when a fluorescent signal or other measurable signal (e.g., heat or radioactivity) is detected. Examples of such devices are provided by PCT/US02/38852, filed Dec. 3, 2003, as well as U.S. Application Publication Nos. 20030103995 (Ser. No. 10/163,744, filed Jun. 4, 2002) and 20030082105 (Ser. No. 10/215,958, filed Aug. 9, 2002).

### Conjugate Imaging

The ability to evaluate specific protease activity in vivo would thus have considerable clinical and basic science applications. For example, protease imaging could be used to improve the early detection of diseases, to image the efficacy of protease inhibitors, to serve as an in vivo screening tool for drug development, and to understand how protease activities are regulated in intact micro- and macro-environments.

Conjugates of the invention can be used for various imaging techniques known in the art. For fluorescence imaging, the optimum excitation and emission wavelength ranges from 650 to 900 nm, because in this near-infrared (NIR) window tissues provides low absorption and low autofluorescence enabling deep penetration with high signal-to-noise ratios. The detection of fluorescence in vivo can be achieved by several techniques, all requiring the use of sensitive devices to detect the small number of photons that are transmitted through tissue. Sensitive NIR animal imaging systems including fluorescent reflectance imaging and fluorescence mediated tomography systems have been developed.

The molar amount of a fluorochrome on a conjugate can be determined by one of ordinary skill in the art using any suitable technique. For example, the molar amount can be determined readily by near infrared absorption measurements. Alternatively, it can be determined readily by measuring the loss of reactive linking groups on the backbone (or spacers), e.g., decrease in ninhydrin reactivity due to loss of amino groups. Following quenching, “de-quenching,” i.e., fluorescence, upon exposure to an activating enzyme is verified in vitro.

Optical image acquisition and image processing can be applied in the practice of the invention. For a review of optical imaging techniques, see, e.g., Alfano et al., 1997, “Advances in Optical Imaging of Biomedical Media,” Ann. NY Acad. Sci. 820:248-270.

An imaging system useful in the practice of this invention typically includes three basic components: (1) a near infrared light source, (2) a means for separating or distinguishing fluorescence emissions from light used for fluorochrome illumination or excitation, and (3) a detection system.

The light source provides monochromatic (or substantially monochromatic) near infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., 1994, Proc. Natl. Acad. Sci. USA 91:4887-4891; Ntziachristos et al., 2000, Proc. Natl. Acad. Sci. USA 97:2767-2772; Alexander, 1991, J. Clin. Laser Med. Surg. 9:416-418.

A high pass filter (700 nm) can be used to separate fluorescence emissions from excitation light. A suitable high pass filter is commercially available from Omega Optical.

In general, the light detection system can be viewed as including a light gathering/image forming component and a light detection/image recording component. Although the light detection system may be a single integrated device that incorporates both components, the light gathering/image forming component and light detection/image recording component will be discussed separately.

A particularly useful light gathering/image forming component is an endoscope. Endoscopic devices and techniques which have been used for in vivo optical imaging of numerous tissues and organs, including peritoneum (Gahlen et al., 1999, J. Photochem. Photobiol. B 52:131-135), ovarian cancer (Major et al., 1997, Gynecol. Oncol. 66:122-132), colon (Mycek et al., 1998, Gastrointest. Endosc. 48:390-394; Stepp et al., 1998, Endoscopy 30:379-386) bile ducts (Izuishi et al., 1999, Hepatogastroenterology 46:804-807), stomach (Abe et al., 2000, Endoscopy 32:281-286), bladder Kriegmair et al., 1999, Urol. Int. 63:27-31; Riedl et al., 1999, J. Endourol. 13:755-759), and brain (Ward, 1998, J. Laser Appl. 10:224-228) can be employed in the practice of the present invention.

Other types of light gathering components useful in the invention are catheter-based devices, including fiber optics devices. Such devices are particularly suitable for intravascular imaging. See, e.g., Tearney et al., 1997, Science 276:2037-2039; Proc. Natl. Acad. Sci. USA 94:4256-4261.

Still other imaging technologies, including phased array technology (Boas et al., Proc. Natl. Acad. Sci. USA 91:4887-4891, 1994; Chance, Ann. NY Acad. Sci. 838:29-45, 1998), optical tomography (Cheng et al., Optics Express 3:118-123, 1998; and Siegel et al., Optics Express 4:287-298, 1999), intravital microscopy (Dellian et al., Br. J. Cancer 82:1513-1518, 2000; Monsky et al, Cancer Res. 59:4129-4135, 1999; and Fukumura et al., Cell 94:715-725, 1998), confocal imaging (Korlach et al., Proc. Natl. Acad. Sci. USA 96:8461-8466, 1999; Rajadhyaksha et al., J. Invest. Dermatol. 104:946-952, 1995; and Gonzalez et al., J. Med. 30:337-356, 1999) and fluorescence molecular tomography (FMT) (Nziachristos et al., Nature Medicine 8:757-760, 2002; U.S. Pat. No. 6,615,063, PCT Application No. WO 03/102558, and PCT US/03/07579) can be used with the fluorochrome conjugates of the invention. Similarly, the fluorochrome conjugates can be used in the IVIS® Imaging System (Xenogen, Alameda, Calif.), the SoftScan® or the eXplore Optix™ (Advanced Research Technologies, Montreal, Canada) system.

Any suitable light detection/image recording component, e.g., charge coupled device (CCD) systems or photographic film, can be used in the invention. The choice of light detection/image recording will depend on factors including type of light gathering/image forming component being used. Selecting suitable components, assembling them into a near infrared imaging system, and operating the system is within ordinary skill in the art.

In some embodiments of the invention, two (or more) conjugates containing: (1) fluorochromes that absorb and/or emit fluorescence at different wavelengths, and (2) activation sites recognized by different enzymese, e.g., cathepsin D and MMP-2, are used simultaneously. This allows simultaneous evaluation of two (or more) biological phenomena.

In some embodiments of the invention, one conjugate contains two or more fluorochromes that absorb and/or emit fluorescence at different wavelengths, wherein at least one fluorochrome is a photosenstizer, and the same or different activation sites. This allows for multi-channel imaging and photoactivation, wherein one can image using one illumination and detection step and one can photoactivate using another distinct illumination and detection step.

The invention features an in vivo optical imaging method. The method includes: (a) administering to a subject fluorochrome conjugate of the invention to a subject; (b) allowing the fluorochrome conjugate to distribute within the subject; (c) illuminating the subject to light of a wavelength absorbable by the fluorochromes of the fluorochrome conjugate; and (d) detecting an optical signal emitted by the fluorochrome. The signal emitted by the fluorochrome can be used to construct an image, either alone or as fused (combined or composite) images with other imaging modalities, including but not limited to magnetic resonance, ultrasound, X-ray, and computed tomography images. In certain embodiments, steps (a) through (d) above further comprise the step of activating the fluorochrome conjugate within the subject prior to step (c).

The above methods can be used, e.g., for in vivo imaging of a tumor in a human patient. The invention also features an in vivo method for selectively imaging two different cells, targets or tissue types simultaneously.
(a) administering to a subject one or more fluorochrome conjugates of the invention, said one or more fluorochrome conjugates comprising at least two fluorochomes which emit distinct wavelengths of light upon illumination;
(b) allowing said one or more fluorochrome conjugates to distribute within the subject;
(c) illuminating the subject with light of a wavelength sufficient to be absorbed by the fluorochromes of said one or more fluorochrome conjugates; and
(d) detecting the optical signals emitted by said fluorochromes.
   The signal emitted by the fluorochromes can be used to construct an image, either alone or as fused (combined or composite) images with other imaging modalities, including but not limited to magnetic resonance, ultrasound, X-ray, and computed tomography images. In certain embodiments, steps (a) through (d) above further comprise the step of activating the fluorochrome conjugate within the subject prior to step (c). Each of the two conjugates comprises a fluorochrome whose fluorescence wavelength is distinguishable from that of the other fluororochrome, and each of the two conjugates optionally contain a different activation site.

### Treatment of Disorders

In certain embodiments, the invention provides a photodynamic therapeutic agent, wherein at least one of the photsensitizer has excitation and emission wavelengths between 500 nm and 1300 nm. In another embodiment, the invention provides a photodynamic therapeutic agent, wherein the photodynamic therapeutic agent is biocompatible.

In certain aspects, the invention provides a composition for treating a subject for neoplastic, vascular, infectious, degenerative, and autoimmune disorders, comprising, a polymeric backbone; a substrate for a targeted moiety; at least one spacer molecule; at least one solubility enhancing group; and at least one photosensitizer covalently linked to the backbone at optical-quenching interaction-permissive positions separable by enzymatic cleavage at activation sites; wherein the photosensitizer generates oxygen radicals. In preferred embodiments, the backbone is a dendrimer.

In one aspect, the invention provides an in vivo photodynamic therapy method comprising: (a) administering to a subject the photodynamic therapeutic agent of the invention; (b) allowing time for the photodynamic therapeutic agent to accumulate in a desired area in the subject; (c) illuminating the subject with light of a wavelength absorbable by the photosensitizers; resulting in cytotoxic singlet oxygen generation; and optionally, (d) detecting fluorescence emitted by the photosensitizers.

The invention features an in vivo optical imaging and photodynamic therapy method. The method includes:

(a) administering to a subject a fluorochrome conjugate of the invention, wherein at least one fluorochrome is a photosenstizer having optical properties distinct from the other fluorochromes;

(b) allowing the fluorochrome conjugate to distribute within the subject;

(c) illuminating the subject with light of a wavelength sufficient to be absorbed by the non-photosensitizer fluorochromes of the fluorochrome conjugate;

(d) detecting an optical signal emitted by the non-photosensitizer fluorochromes;

(e) illuminating the subject with a second light of a wavelength sufficient to produce cytotoxic singlet oxygen by the photosensitizer; and

(f) detecting fluorescence emitted by the photosensitizers. In certain embodiments, steps (a) through (f) above further comprise the step of activating the fluorochrome conjugate within the subject prior to step (c).

In certain embodiments, the photodynamic therapeutic agent is activated by cathepsins, matrix metalloproteinases (MMP), membrane-type MMPs, collagenases, gelatinases, stromelysins, caspases, viral proteases, HIV proteases, HSV proteases, gelatinase, urokinases, secretases, endosomal hydrolase, Prostate Specific Antigen (PSA), plasminogen activator, Cytomegalovirus (CMV) protease, or thrombin. In a further embodiment, the photodynamic therapeutic agent is activated by Cathepsin S. In a further embodiment, the at least one photosensitizer that has excitation and emission wavelengths between 500 nm and 1300 nm. In another embodiment, steps (a) through (d) supra, are repeated over time. In certain embodiments, the fluorescence emitted from step (d) is used to construct an image. In another embodiment, step (d) supra is performed using a suitable light detection or image recording component consisting of a charge coupled device (CCD) system or photographic film. In other embodiments, steps (c) and (d) supra, are performed using an endoscopic device, a catheter-based device, a diffuse optical tomographic imaging system, phased array technology, confocal imaging, intravital microscopy or intraoperative imaging device. In another embodiment, the presence, absence or level of photodynamic therapeutic agent activation is indicative of a disease state.

In certain embodiments, the disease state is cancer, tumors, tumor progression, tumor growth, neoplastic disease, neovascularization, cardiovascular disease, angiogenesis, intravasation, extravasation, metastasis, apoptosis, arthritis, infection, HIV infection, HSV infection, Alzheimer's Disease, blood clotting, atherosclerosis, leukemia, lymphoma, melanoma, osteosarcoma, or osteoporosis. In certain embodiments, the disease state is cancer. In other embodiments, the subject is a living animal, preferably a human.

In other aspects, the invention provides a method for treating a subject for neoplastic, vascular, infectious, degenerative, and autoimmune disorders, comprising administering to said subject in need thereof an effective amount of an intramolecularly-quenched photodynamic therapeutic agent comprising, a polymeric dendrimer-based backbone; a substrate for a targeted moiety; at least one spacer molecule; at least one solubility enhancing group; and at least one photosensitizer covalently linked to the dendrimer backbone at optical-quenching interaction-permissive positions separable by enzymatic cleavage at activation sites; wherein the complex generates oxygen radicals, such that said subject is treated for said disorder.

In one embodiment, the invention further comprises activation of the complex by administration of light between 500 and 800 nm. In other embodiments, the disorder is cancer, tumors, tumor progression, tumor growth, neoplastic disease, neovascularization, cardiovascular disease, angiogenesis, intravasation, estravasation, metastasis, apoptosis, arthritis, infection, HIV infection, HSV infection, alzheimer's disease, blood clotting, atherosclerosis, leukemia, lymphoma, melanoma, osteosarcoma, or osteoporosis. In certain embodiments, the disorder is cancer, tumors, tumor progression, tumor growth, or neovascularization. In a further embodiment, the disorder is colon cancer, lung cancer, esophagus cancer, genitourinary tract cancer, brain cancer, ovary cancer, rectal cancer, prostate cancer, bladder cancer, or breast cancer. In other further embodiments, the disorder is a malignant tumor of the skin, esophageal, lung, breast, gastrointestinal tract, genitourinary tract, bladder, or cervix.

In certain embodiments, there is little or no toxicity associated with conjugate solubility, membrane accumulation, or non-specific binding, due to the self quenching. In other embodiments, the oxygen radicals generated by the conjugate directly destroy the cells related to said disorder. In certain embodiments, the oxygen radicals generated by the conjugate affect the vascular supply of said disorder.

In another aspect, the invention provides a method for treating a subject for neoplastic, vascular, infectious, degenerative, and autoimmune disorders, comprising administering to said subject in need thereof an effective amount of an intramolecularly-quenched photodynamic therapeutic agent comprising, a polymeric dendrimer-based polylysine backbone; a Leu-Arg substrate for a targeted moiety, wherein the targeted moiety is cathepsin S.; at least one β-alanine spacer molecules; at least one PEG solubility enhancing groups; at least one chlorin e6 covalently linked to the dendrimer polylysine backbone at optical-quenching interaction-permissive positions separable by enzymatic cleavage at activation sites; wherein the combination of photosensitizer and solubility enhancing groups together provides a dye wherein said dye is: Ce6PEG-1, Ce6PEG-2, or Ce6PEG-3; and wherein the complex generates oxygen radicals; such that said subject is treated for said disorder.

In one embodiment, the invention provides a method for treating a disorder, further comprising surgery. In another embodiment, the invention a method for treating a disorder, further comprising chemotherapy. In another embodiment, the invention a method for treating a disorder, further comprising additional radiation therapy. In certain embodiments, the subject is a mammal, preferably a human.

### Formulation and Administration

The invention also provides a pharmaceutical composition, comprising an effective amount a conjugate described herein and a pharmaceutically acceptable diluent or carrier. In certain embodiments, the conjugate is administered to the subject in a pharmaceutically-acceptable formulation. In certain embodiments, the pharmaceutical compositions are suitable for topical, intravenous, intratumoral, parental, or oral administration. The methods of the invention further include administering to a subject a therapeutically effective amount of a conjugate in combination with another pharmaceutically active compound. Pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference.

The phrase “pharmaceutically acceptable” refers to conjugates of the present invention, compositions containing such conjugates, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, involved in carrying or transporting the subject chemical from one organ, or portion of the body, to another organ, or portion of the body. Each carrier is “acceptable” in the sense of being compatible with the other ingredients of the formulation and not injurious to the patient.

Methods of preparing these compositions include the step of bringing into association a conjugate with the carrier and, optionally, one or more accessory ingredients. These compositions may also contain adjuvants such as preservatives, wetting agents, emulsifying agents and dispersing agents.

Regardless of the route of administration selected, the conjugates, which may be used in a suitable hydrated form, and/or the pharmaceutical compositions of the present invention, are formulated into pharmaceutically-acceptable dosage forms by conventional methods known to those of skill in the art.

Formulations are provided to a subject in an effective amount. The term “effective amount” includes an amount effective, at dosages and for periods of time necessary, to achieve the desired result. An effective amount of conjugate may vary according to factors such as the disease state, age, and weight of the subject, and the ability of the compound to elicit a desired response in the subject. Dosage regimens may be adjusted to provide the optimum therapeutic response.

The effective amount is generally determined by the physician on a case-by-case basis and is within the skill of one in the art. As a rule, the dosage for in vivo therapeutics or diagnostics will vary. Several factors are typically taken into account when determining an appropriate dosage. These factors include age, sex and weight of the patient, the condition being treated, and the severity of the condition.

Suitable dosages and formulations of immune modulators can be empirically determined by the administering physician. Standard texts, such as Remington: The Science and Practice of Pharmacy, 17th edition, Mack Publishing Company, and the Physician's Desk Reference, each of which are incorporated herein by reference, can be consulted to prepare suitable compositions and doses for administration. A determination of the appropriate dosage is within the skill of one in the art given the parameters for use described herein.

Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

In terms of treatment, an effective amount is an amount that is sufficient to palliate, ameliorate, stabilize, reverse or slow the progression of a cancerous disease (e.g. tumors, dysplaysias, leukemias) or otherwise reduce the pathological consequences of the cancer. A therapeutically effective amount can be provided in one or a series of administrations. In terms of an adjuvant, an effective amount is one sufficient to enhance the immune response to the immunogen. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the antibody being administered.

The dosage of the conjugates can vary from about 0.01 mg/m² to about 500 mg/m², preferably about 0.1 mg/m² to about 200 mg/m², still more preferably about 0.1 mg/m² to about 10 mg/m². In other embodiments, the complex is administered at a concentration of 0.001 μg-1 mg/kg of body weight. Ascertaining dosage ranges is well within the skill of one in the art. The dosage of conjugates can range from about 0.1 to 10 mg/kg. Methods for administering photosensitize compositions are known in the art, and are described, for example, in U.S. Pat. Nos. 5,952,329, 5,807,881, 5,798,349, 5,776,966, 5,789,433, 5,736,563, 5,484,803 and by (Sperduto et al., 1991), (Walther et al., 1997). Such dosages may vary, for example, depending on whether multiple administrations are given, tissue type and route of administration, the condition of the individual, the desired objective and other factors known to those of skill in the art. Administrations can be conducted infrequently, or on a regular weekly basis until a desired, measurable parameter is detected, such as diminution of disease symptoms. Administration can then be diminished, such as to a biweekly or monthly basis, as appropriate.

A therapeutically effective amount can be administered in one or more doses. The term “administration” or “administering” includes routes of introducing the compound(s) to a subject to perform their intended function. Examples of routes of administration which can be used include injection (subcutaneous, intravenous, parenterally, intraperitoneally, intrathecal), oral, inhalation, rectal and transdermal.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

Such dosages may vary, for example, depending on whether multiple administrations are given, tissue type and route of administration, the condition of the individual, the desired objective and other factors known to those of skill in the art. Where the conjugates comprises a photosensitizer conjugated to an antibody.

Following administration of the conjugate, it can be necessary to wait for the conjugate to reach an effective tissue concentration at the site of the disorder before detection. Duration of the waiting step varies, depending on factors such as route of administration, location, and speed of movement in the body. In addition, where the compositions are coupled to molecular carriers, the rate of uptake can vary, depending on the level of receptor expression on the surface of the cells. For example, where there is a high level of receptor expression, the rate of binding and uptake is increased. Determining a useful range of waiting step duration is within the level of ordinary skill in the art and may be optimized by utilizing fluorescence optical imaging techniques.

Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with other pharmaceutical agents. Photosensitizers are often administered by injection or by gradual perfusion.

### Oral Dosage Forms

Conjugates of the invention and compositions comprising them that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent particularly advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103.TM and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention.

The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Parenteral and Intravascular Dosage Forms

Parenteral and intravascular dosage forms can be administered to patients by various routes including, but not limited to, subcutaneous, intravenous (including bolus injection and constant infusion), intramuscular, and intraarterial. Because their administration typically bypasses patients' natural defenses against contaminants, parenteral and intravascular dosage forms are preferably sterile or capable of being sterilized prior to administration to a patient. Examples of parenteral dosage forms include, but are not limited to, solutions ready for injection, dry products (including, but not limited to lyophilized powders, pellets, and tablets) ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

For intravascular administration, for instance by direct injection into the blood vessel, or surrounding area, it may be desirable to administer the compositions locally to the area in need of treatment. This can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel provided by Guilford Pharmaceuticals Inc.

### Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds.; Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Administration Considerations

Following administration of the conjugate, it is typically necessary to wait for the photosensitizer to reach an effective tissue concentration at the site before photoactivation. Duration of the waiting step varies, depending on factors such as route of administration, tumor location, and speed of movement in the body. The waiting period should also take into account the rate at which conjugates are degraded and thereby dequenched in the target tissue. Determining a useful range of waiting step duration is within ordinary skill in the art and may be optimized by utilizing fluorescence optical imaging techniques.

Following the waiting step, the conjugate is activated by photoactivating light applied to the target site. This is accomplished by applying light of a suitable wavelength and intensity, for an effective length of time, specifically to the target site. The suitable wavelength, or range of wavelengths, will depend on the particular photosensitizer(s) used. Wavelength specificity for photoactivation depends on the molecular structure of the photosensitizer. Photoactivation occurs with sub-ablative light doses. Determination of suitable wavelength, light intensity, and duration of illumination is within ordinary skill in the art.

The light for photoactivation can be produced and delivered to the target site by any suitable means. For superficial target sites or open surgical sites, suitable light sources include broadband conventional light sources, broad arrays of light emitting diodes (LED), and defocussed laser beams.

For non-superficial lesion sites, including those in intracavitary settings, the photoactivating light can be delivered by optical fiber devices. For example, the light can be delivered by optical fibers threaded through small gauge hypodermic needles. Optical fibers also can be passed through arthroscopes, endoscopes and laproscopes. In addition, light can be transmitted by percutaneous instrumentation using optical fibers or cannulated waveguides.

Photoactivation at non-superficial target sites also can be by transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, i.e. lightboxes or convergent light beams.

For photoactivation, the wavelength of light is matched to the electronic absorption spectrum of the photosensitizer so that photons are absorbed by the photosensitizer and the desired photochemistry can occur. Except in special situations, where the tumors being treated are very superficial, the range of activating light is typically between approximately 600 and 900 nm. This is because endogenous molecules, in particular hemoglobin, strongly absorb light below about 600 nm and, therefore, capture most of the incoming photons (Parrish, 1978). The net effect would be the impairment of penetration of the activating light through the tissue. The reason for the 900 nm upper limit is that energetics at this wavelength may not be sufficient to produce ¹O₂, the activated state of oxygen, which without wishing to necessarily be bound by any one theory, is perhaps very important for successful PDT. In addition, water begins to absorb at wavelengths greater than about 900 nm. While spatial control of illumination provides specificity of tissue destruction, it can also be a limitation of PDT. Target sites are advantageously accessible to light delivery systems, and issues of light dosimetry are advantageously addressed (Wilson, 1989). In general, the amenability of lasers to fiberoptic coupling makes the task of light delivery to most anatomic sites manageable, although precise dosimetry remains complex and elusive.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (e.g., 700-800 nm) (Svaasand and Ellingsen, 1983). These values can be altered by altering the biologic interactions and physical characteristics of the photosensitizer. Factors such as self-shielding and photobleaching (self-destruction of the photosensitizer during the PDT) further complicate precise dosimetry. In general, photosensitizers with longer absorbing wavelengths and higher molar absorption coefficients at these wavelengths are more effective photodynamic agents.

PDT dosage depends on various factors, including the amount of the photosensitizer administered, the wavelength of the photoactivating light, the intensity of the photoactivating light, and the duration of illumination by the photoactivating light. Thus, the dose of PDT can be adjusted to a therapeutically effective dose by adjusting one or more of these factors. Such adjustments are within ordinary skill in the art.

Although methods and materials similar or equivalent to those described herein can be used in the practice of the present invention, preferred methods and materials are described below. The materials, methods, and examples are illustrative only and not intended to be limiting. Other features and advantages of the invention will be apparent from the detailed description and from the claims.

### Kits

This invention therefore encompasses kits which, when used by the medical practitioner, can simplify the administration of appropriate amounts of conjugates of the invention to a patient.

A typical kit of the invention comprises one or more unit dosage forms of a conjugate of the invention, and instructions for use.

Kits of the invention can further comprise devices that are used to administer fluorescent conjugates of the invention. Examples of such devices include, but are not limited to, intravenous cannulation devices, syringes, drip bags, patches, topical gels, pumps, containers that provide protection from photodegredation, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Kits of the invention can further compromise devices that facilitate the illumination of a fluorochrome conjugate of a particular wavelength of light including broadband conventional light sources, light-emitting diodes (LEDs), defocused lasers, light guides, fiber optic transmitters, and other such devices.

The invention is further described by way of the following non-limiting examples.

### EXAMPLES

In order that the invention may be more fully understood, the following examples are provided. It should be understood that these examples are for illustrative purposes only and are not to be construed as limiting the invention in any way.

### Example 1

### Solid-Phase Peptide Synthesis

A series of exemplary conjugates with four (CyPEG-1), eight (CyPEG-2) and twelve (CyPEG-3) ethylene oxide units in the dendritic arms were synthesized by solid-phase methods to determine the optimum PEG chain length that can improve the aqueous solubility of the conjugate but at the same time does not weaken dye aggregation (FIG. 2). Fifty mg of Fmoc₄-Lys₂-Lys-β-Ala Wang Resin (100-200 mesh), purchased from Peptides International (Louisville, Ky.) was added into the 6 mL capacity single fritted reservoir (Biotage-Argonaut, Redwood City, Calif.) and shaken with 3.5 mL of dichloromethane (DCM) for 15 min. A tetrapeptide (β-Ala-Leu-Arg(Pbf)-β-Ala) was built on this solid support manually by using the following protocol. Fmoc deprotection: 2 cycles of 20 min each with 3.5 mL of 20% piperidine in N,N-dimethylformamide (DMF) followed by washing of the resin with DMF (4×3 mL); amino acid coupling: 5 equivalents (with respect to the loading level of the resin) of the fluorenylmethoxycarbonyl (Fmoc) protected amino acids (Novabiochem, San Diego, Calif.), 2-(1-H-Benzotriazole-1-yl)-1,1,3,3-tetramethyluronium hexafluorophosphate (HBTU), and N-Hydroxybezotriazole (HOBt) were dissolved in 3 mL of peptide synthesis grade DMF in a small glass beaker and 100 μL of N,N-diisopropylethylamine (DIEPA) was added. The solution was allowed to stand for 15 min and was added to the resin in a single fritted reservoir. After tightly securing the reservoir, the suspension was shaken overnight. The resin was then washed four times with DMF, and three times with DCM. All coupling reactions were monitored by the ninhydrin and chloranil tests using a dry resin sample.

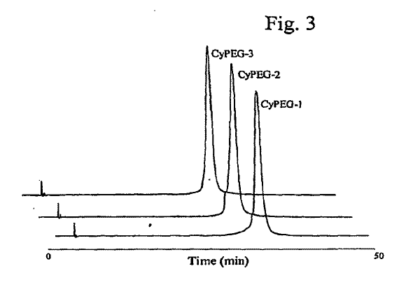

The bifunctional PEG chain utilized in this design had no polydispersity. Thus, each conjugate was a single chemical entity. Homogeneity of the conjugates was confirmed using RP-HPLC (FIG. 3) and their masses were confirmed using ESI-MS (Table 3). It should be noted that conjugates of the invention can be confirmed using any analytical methods known to those of skill in the art including, but not limited to, those techniques listed above as well as HPLC, HPLC-MS, NMR spectroscopy, UV spectroscopy, and IR spectroscopy.

TABLE 3
Characterization data for CyPEG-1, CyPEG-2, and CyPEG-3.

| | Molecular mass | Molecular mass | Retention |
|---|---|---|---|
| Probe | (calculated)^{a} | (found)^{b} | time^{c} |
| CyPEG-1 | 6224.1 Da | 6225.6 Da | 31.99 min. |
| CyPEG-2 | 7212.7 Da | 7213.2 Da | 30.63 min. |
| CyPEG-3 | 8201.2 Da | 8201.5 Da | 29.46 min. |

^{a}Theoretical molecular mass expected for the molecular ion M⁺
^{b}molecular mass found from the ESI-MS for the molecular ion M⁺ ionic species.
^{c}See the experimental section for details of the conditions used in the RP-HPLC.

Wavelengths (λₘₐₓ) of fluorescence excitation and emission of CyTE-777 did not change after its conjugation to the dendritic scaffold. On-resin synthesis typically requires excess of reagents to drive the reactions to completion. CyTE-777 affords access to a variety of constructs based on solid-phase synthesis.

### Example 2

### PEG Coupling

Five equivalent (80 mg) of Fmoc-15-amino-4,7,10,13-tetraoxapentadecanoic acid (NeoMPS, Strasbourg, France) and 110 mg of Benzotriazole-1-yl-oxy-tris-pyrrolidino-phosphonium hexafluorophosphate (PyBOP), were dissolved in a 3 mL solution of 9:1 N-methyl-2-pyrrolidone (NMP)/DCM in a small glass beaker and 60 μL of DIEPA was added. The solution was allowed to stand for 30 min and was added to the resin-bound tetrapeptide. The Fmoc group was deprotected as mentioned above. This protocol of PEG coupling and Fmoc deprotection was repeated once for CyPEG-2 and twice for CyPEG-3. All the couplings were monitored by the ninhydrin and chloranil tests using a dry resin sample.

### Example 3

### Dye Conjugation

Following PEG coupling and Fmoc deprotection, the peptide resin was collected for further coupling of the NIR dye. Fifty mg of CyTE-777 (5 equiv.) and 10 mg of HOBt were dissolved in 2 mL of anhydrous DMF and chilled to 0° C. To this, 30 mg of N,N′-dicyclohexylcarbodiimide (DCC) was added and dissolved by gentle stirring. The solution was then warmed to the room temperature and allowed to stand for 60 min after which it was added to the resin-bound pegylated peptide.

### Example 4

### Cleavage and Purification

Following dye conjugation, the resin was washed 5 times with DMF and 3 times with DCM and dried under vacuum. The dried resin sample (−50 mg) was treated with 3 mL solution of 15:1 trifluoroacetic acid (TFA)/triisopropylsilane (TIS) for 2 h. The resin suspension was filtered into 30 mL of hexanes and the solvents were removed in vacuo at 45° C. using a water aspirator. The dried sample was dissolved in 20 mL of 1:1 water/acetonitrile and lyophilized to obtain dry powder. Lyophilization was repeated twice. All the probes were purified by reversed phase-high performance liquid chromatography (RP-HPLC) using a Vydac preparative column (C18, 10 μm, 22 mm ID×250 mm L, Hesperia, Calif.) on a Hitachi D-7000 HPLC system equipped with a Hitachi L-7100 pump and a Hitachi L-7455 diode array detector. HPLC conditions: Buffer A—0.1% TFA in water, Buffer B—0.1% TFA in acetonitrile; solvent gradient—0% B to 100% B in 50 min; flow rate—6 mL/min; UV detection—220 nm. The overall yield of synthesis was between 40 to 42% for all the three probes.

### Example 5

### Optical Properties of the Photosensitizer Conjugates

The optical properties (excitation and emission) of photosensitizers are studied using spectrophotometry (Cary 50, Varian, Palo Alto, Calif.) and fluorescence spectrophotometry (Fluorolog-3, Horiba Jobin Yvon, Edison, N.J.) before and after coupling reactions. For these experiments, the photosensitizer conjugates (0.1 mg) is dissolved in 2 ml of PBS buffer, and the molarity of the photosensitizer is calculated by absorbance. A standard solution with same concentration of free photosensitizer is prepared for calibration. The quenching efficiency is calculated by dividing the fluorescence emission signal of the quenched probe to that of free photosensitizer. Briefly, stock solutions of the photosensitizers with optical densities of 0.03, as well as a 0.25 M solution of 1,3-diphenylisobenzofuran (DPBF), all in DMF, are mixed and kept in the dark. Into a fluorescence cuvette, 2.0 ml of the stock photosensitizer solution is added containing the DPBF solution (8 μL, final concentration, 1 mM) before irradiation at 650 nm (60 mW) in a fluorescence spectrophotometer under constant stirring. Simultaneously, the fluorescence emission intensity of DPBF is monitored (excitation 471 nm, emission 495 nm). Singlet oxygen quantum yields are then calculated from the initial slope of the fluorescence intensity decrease utilizing the following equation:

Φ_{Δ}(U)=Φ_{Δ}(St)×S(U)/S(St)

Where U and St denote unknown and standard, and S represents the slope. Chlorin e6 is used as the standard.

### Example 6

### Quenching

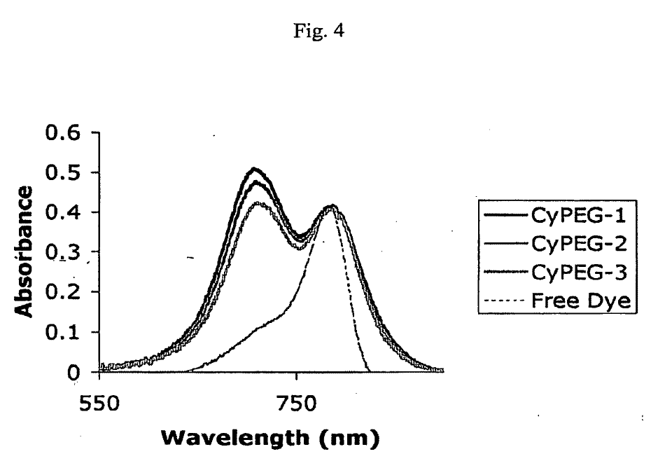

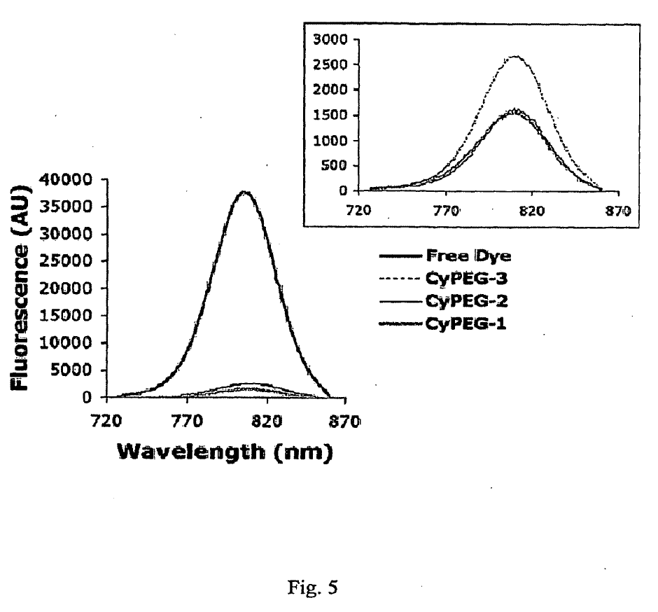

The absorption spectra of equimolar solutions of the probes based on the absorption peak of the free dye, CyTE-777 are shown in FIG. 4. A new peak at 705 nm, which is blue-shifted by about 80 nm from the absorption peak of the free dye was observed for all the three probes indicating dye aggregation. At this concentration (3 μM), free CyTE-777 does not show any aggregation in water or aqueous buffered solutions. However after its conjugation to the branched lysine core, the probe displays significant dye aggregation thus demonstrating the ability of the branched lysine scaffold to promote aggregation. The absorption band due to dye aggregation becomes slightly weaker with increasing PEG chain length. CyPEG-1, with the strongest dye-dye aggregation, is practically insoluble in aqueous solutions. CyPEG-2 and CyPEG-3 on the other hand showed satisfactory aqueous solubility. To determine if the aggregated fluorophores lead to quenching, fluorescence spectra of all the three probes were measured and compared to the fluorescence spectrum of an equimolar solution of CyTE-777 (FIG. 5). All the probes displayed superior quenching efficiency with CyPEG-1 (95.62%) and CyPEG-2 (95.57%) showing better quenching than CyPEG-3 (92.50%).

Typically, in a homolabeled probe, two possible mechanisms are suggested to explain the quenching phenomenon. First is the fluorescence resonance energy transfer (FRET), based on a weak dipole-dipole interaction and second is static quenching, based on strong dipole-dipole interactions, where the fluorochromes form non-fluorescent ground-state complexes (Packard, B. Z. et al. Methods Enzymol 1997., 278, 15-23). In FRET-based quenching, the fluorophore and quencher molecules retain their intrinsic properties such as the absorption spectrum. Static quenching, however, is associated commonly with significant changes in the absorption spectra. If the interaction between the two dyes leads to a new blue-shifted absorption maximum then the type of association is termed H-dimer, an alignment between the two dyes such that a radius vector connecting the two chromophores is perpendicular to their transition dipoles. Such association typically produces weakly fluorescent complexes with their own distinct absorption spectra. In homodimers it is possible for an H-type aggregate to have no fluorescence because identical transition dipoles are coupled which can completely cancel each other if properly aligned. In such a dimeric state, absorption and emission of light are by the dimeric unit.

This mechanism is quite different from the FRET. In a heterolabeled probe, the spectral overlap between a donor and an acceptor gives rise to absorption of light by the donor chromophore, resonance energy transfer to the acceptor chromophore, and emission with the spectral characteristics of the acceptor exclusively. In a homolabeled probe, however, each chromophore has a dual role of being a donor as well as an acceptor.

H-dimers typically have diminished fluorescence and as indicated in FIG. 4 all the probes showed substantial decrease in fluorescence as compared to their free dye counterpart at the same concentration. As indicated in FIG. 4, the absorption spectra of all the three probes showed a substantial aggregation peak (705 nm). Formation of H-dimers, confirmed by the absorption and fluorescence spectra suggest static quenching of CyTE-777 in the probes.

However, FRET-based quenching of the fluorophore is also feasible because CyTE-777 has a relatively small Stoke's shift 30 nm). The small Stoke's shift results in significant overlap between the absorption and the emission spectra of CYTE-777. This overlap facilitates FRET and subsequent quenching of the fluorophore. FRET typically occurs over distances up to 20-60 Å. In a MAP-based system, the dye molecules are in close association with each other, and FRET is expected for the molecules that do not form H-dimeric complexes. Thus, in the MAP-based design of a fluorescent quenched probe, the contribution from the FRET-based quenching of the probe cannot be ruled out.

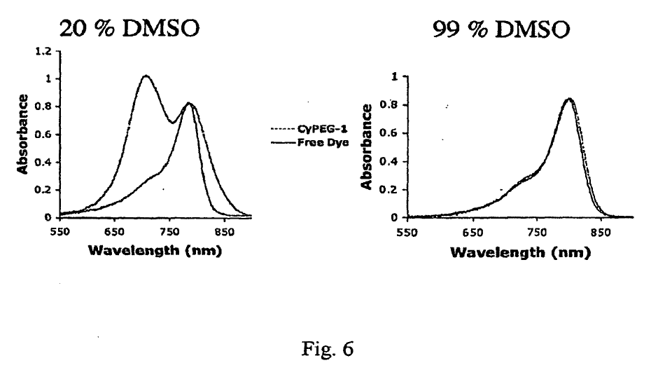

To investigate the contribution of FRET to the quenching of the probe, the dye-dye aggregation and static quenching mechanisms were eliminated by alteration of the solvent polarity. Equimolar solutions (6.5 μM) were made for CyPEG-1 and CyTE-777 and the absorption spectra and fluorescence quenching of CyPEG-1 monitored and compared to the free dye in 20% and 99% DMSO (FIG. 6). As compared to free CyTE-777, the blue-shifted aggregation peak of CyPEG-1 observed in 20% DMSO can be entirely eliminated by dissolving the probe in 99% DMSO. Thus, in 99% DMSO, there is little or no contribution from formation of non-fluorescent ground state complexes on the quenching of CyPEG-1. To evaluate the contribution of FRET on the quenching of CyPEG-1, fluorescence of CyTE-777 and CyPEG-1 in 20% DMSO and 99% DMSO was measured. In 20% DMSO, the fluorescence emission of the free dye is approximately 38-fold greater than that observed for CyPEG-1. In 99% DMSO, where there is negligible contribution from static quenching in CyPEG-1, the fluorescence emission from the free dye is only 1.7 times that of CyPEG-1 indicating static quenching as the predominant mechanism in the MAP-based system of the probes. Probes based on static quenching represent a significant advantage over FRET-based probes because in homolabeled probes where static quenching mechanism predominates, an intrinsically more favorable signal to noise ratio is obtained in which the signal itself is truly fluorogenic.

### Example 7

### Enzymatic Studies

The protease sensitive probes exemplified herein are designed to be activated by cathepsin S. Among 11 cysteine cathepsins present in the human genome, cathepsin S is unique because of its potential to be active at neutral pH in the extracellular matrix, suggesting its role in invasion (Turk, V. et al. Cancer Cell 2004, 5, 409-4100). High expression of cathepsin S has been found in lung alveolar macrophages (Kos, J. et al. Br. J. Cancer 2001, 85, 1193-1200), brain tumor (Flannery, T. et al. Am. J. Pathol. 2003, 163, 175-182), rheumatoid arthritis (Yasuda, Y. et al. Adv. Drug Deilv. Rev. 2005, 57, 973-993) and atherosclerosis (Liu, J. et al. Arterioscler Thromb Vasc Biol. 2004, 24, 1359-1366).

All the studies were performed in triplicate with 200 μL of the sample in a 96 well assay plate with clear bottom and lid (Corning Inc. NY USA). Fluorescence measurements were obtained in a fully modular monochromator-based microplate detection system (Safire, Tecan, San Jose, Calif.). Enzymes and enzyme inhibitors were purchased from EMD Biosciences (San Diego, Calif.). Concentration of all the conjugates and CyTE-777 were adjusted to ˜3 μM (based on the absorption spectra, O.D. of 0.4) using a solution of 20% dimethylsulfoxide (DMSO) in 10 mM phosphate buffer, pH 7.4.

For all the activation studies 0.55 nmole of probe and 0.16 nmole of enzyme (human recombinant cathepsin S, Human liver cathepsin L, Human recombinant cathepsin K) were mixed. Excitation and emission were set at 750 nm and 810 nm, respectively, with a bandwidth of 20 nm. The fluorescence was monitored for 9 h. at 27° C. Control experiments were performed simultaneously by replacing the enzyme with either 10 mM phosphate buffer or by adding E-64 protease inhibitor (2 nM) to the enzyme.

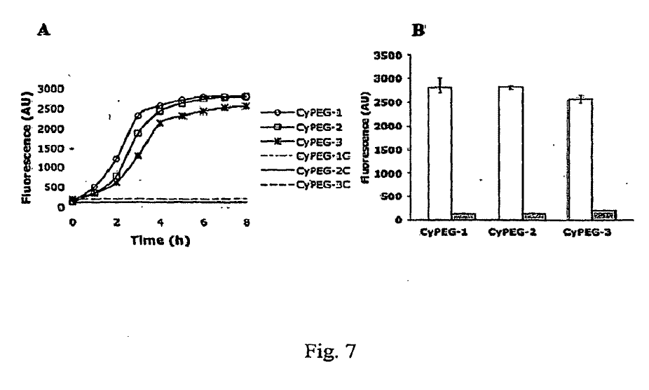

Considering the differential solubility of all the probes, the enzyme activation studies were conducted in the presence of 20% DMSO in 10 mM phosphate buffer solution of pH 7.4. All the probes showed an increase in fluorescence on treatment with cathepsin S (FIG. 7). Enzymatic activation, caused by the proteolytic release of the fluorochromes, slowed down with increase in the length of the PEG chain. After 8 h, fluorescence from all the probes reached saturation. Fluorescence obtained at this time point was compared to the fluorescence of control where no enzyme was added. Of the three probes (CyPEG-1, CyPEG-2, and CyPEG-3), CyPEG-3 displayed the smallest increase in fluorescence signal due to its longer PEG chain length, which results in weaker dye aggregation and, therefore, a higher fluorescence background.

The increase in fluorescence as compared to the background signal is an important factor when evaluating the efficiency of an enzyme activatable probe. Another criterion for the evaluation of the probes is the fluorescence recovery after activation, which is determined by comparing the fluorescence signal of an activated probe after complete enzymatic degradation, with the fluorescence from an equimolar solution of the free fluorochrome. All the three probes showed greater than 95% recovery of fluorescence.

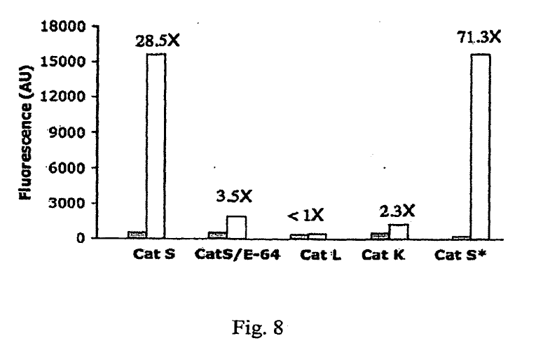

The quenching efficiency and the activation profiles of CyPEG-1 and CyPEG-2 are similar but their aqueous solubility is dramatically different. Because CyPEG-2 has much higher aqueous solubility, this probe is better suited for biological applications. Selectivity studies with CyPEG-2 were performed under optimized pH conditions using several cysteine proteinases, including cathepsin L, cathepsin K, and cathepsin S in 10 mM phosphate buffer solutions of pH 5.5, 4.5, and 6.5, respectively. DMSO (20%) was added to all the buffer solutions to improve the solubility of CyPEG-2 under acidic (pH 5.5 and 4.5) conditions. These cysteine proteases are known to be important in biological processes and have similar substrate selectivity. Cathepsin L and cathepsin K did not show substantial activation of the probe (FIG. 8). The Leu-Arg dipeptide cathepsin S substrate was previously reported to be partially but not completely selective to cathepsin S. The selectivity observed for cathepsin S in the present study is higher than what was expected from the literature reports.

Activation studies of CyPEG-2 with cathepsin S showed more than 70-fold increase in fluorescence in pH 6.5 buffer without DMSO. Even in the presence of 20% DMSO, at pH 6.5, CyPEG-2 still showed 28-fold increase in fluorescence (FIG. 8).

CyTE-777 employed in the design of cathepsin activatable probes has its emission maxima above 800 mu. Thus, fluorescence signal obtained from the activated probe is in the near-infrared (NER) region, which is ideally suited for in vivo imaging. Pegylation of peptides and proteins generally improves their overall pharmacokinetics.

Other studies involving fluorochrome conjugates and their use in photodynamic applications can be found, for example, in “Enzyme-targeted Fluorescent Imaging Probes on a Multiple Antigenic Peptide Core”. J Med. Chem. 2006 Jul. 27; 49(15):4715-20; and Selective Antitumor Effect of Novel Protease-Mediated Photodynamic Agent. Cancer Res. 2006 Jul. 15; 66(14):7225-9.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

1. A fluorochrome conjugate comprising,
a dendrimer;
a protease cleavage site; and
at least two fluorochromes, each covalently linked by a protease cleavage site to the dendrimer at quenching positions.

2. A fluorochrome conjugate comprising,
a backbone;
a protease cleavage site; and
at least two fluorochromes, each covalently linked by a protease cleavage site to the backbone at quenching positions, wherein at least one fluorochrome is a photosentizer.

3. The fluorochrome conjugate of claim 2, wherein the backbone comprises a dendrimer.

4. The fluorochrome conjugate of claim 1, wherein the at least two fluorochromes are photosensitizers.

5. The fluorochrome conjugate of claim 1, further comprising a spacer compound, wherein the spacer compound links a fluorochrome to the dendrimer.

6. The fluorochrome conjugate of claim 1, further comprising at least one solubility enhancing group.

7. The fluorochrome conjugate of claim 1, further comprising at least one targeting moeity.

8. The fluorochrome conjugate of claim 1, wherein the protease cleavage site is in the dendrimer.

9. The fluorochrome conjugate of claim 1, wherein the dendrimer is a branched polypeptide, a branched nucleic acid, a branched polyethyleneamine, a branched polysaccharide, a branched polyamidoamine, a branched polyacrylic acid, a branched polyalcohol or a branched synthetic polymer.

10. The fluorochrome conjugate of claim 9, wherein the dendrimer is a branched polypeptide.

11. The fluorochrome conjugate of claim 10, wherein the branched polypeptide comprises D or L amino acids or a combination thereof.

12. The fluorochrome conjugate of claim 11, wherein the branched polypeptide comprises polylysine.

13. The fluorochrome conjugate of claim 12, wherein the branched polypeptide comprises poly-L-lysine.

14. The fluorochrome conjugate of claim 10, wherein the branched polypeptide comprises albumin.

15. The fluorochrome conjugate of claim 10, wherein the branched polypeptide comprises multiple antigenic peptides.

16. The fluorochrome conjugate of claim 10, wherein the branched polypeptide is an antibody or an antibody fragment.

17. The fluorochrome conjugate of claim 9, wherein the synthetic polymer is polyglycolic acid, polylactic acid, poly(glycolic-colactic) acid, polydioxanone, polyvalero lactone, poly-ε-caprolactone, poly(3-hydroxybutyrate), poly(3-hydroxyvalerate) polytartronic acid, polyasapartic acid, poly glutamic acid, or poly(β-malonic acid).

18. The fluorochrome conjugate of claim 1, wherein the protease cleavage site has an amino acid sequence wherein the sequence is RR, RRG, GPICFFRLG (SEQ. ID. NO. 1), HSSKLQG (SEQ. ID. NO. 2), PIC(Et)FF (SEQ. ID. NO. 3), HSSKLQ (SEQ. ID. NO. 4), P(L/Q)G(I/L)AG (SEQ. ID. NO. 5), GVVQASCRLA (SEQ. ID. NO. 6) or KK.

19. The fluorochrome conjugate of claim 1, wherein the protease cleavage site has an amino acid sequence of Leu-Arg.

20. The fluorochrome conjugate of claim 1, wherein the protease cleavage site is cleaved by a protease wherein the protease is a cathepsin, matrix metalloproteinase (MMP), collagenase, gelatinase, stromelysin, caspase, viral protease, HIV protease, HSV protease, gelatinase, urokinase, secretase, endopeptidase, endosomal hydrolase, or Cytomegalovirus (CMV) protease.

21. The fluorochrome conjugate of claim 20, wherein the cathepsin is Cathepsin B, Cathepsin D, Cathepsin H, Cathepsin K, Cathepsin L, or Cathepsin S.

22. The fluorochrome conjugate of claim 5, wherein the spacer molecule is a peptide, oligopeptide, polysaccharide, a nucleic acid, or a synthetic cleavable moiety.

23. The fluorochrome conjugate of claim 22, wherein the spacer molecule is a peptide.

24. The fluorochrome conjugate of claim 23, wherein the peptide is comprised of glycine or β-alanine.

25. The fluorochrome conjugate of claim 6, wherein the solubility enhancing group links a fluorochrome to a spacer compound.

26. The fluorochrome conjugate of claim 6, wherein the solubility enhancing group links at least one fluorochrome to the protease cleavage site of the peptide.

29. The fluorochrome conjugate of claim 25, wherein the solubility enhancing group is polyethylene glycol (PEG), methoxypolyethylene glycol (MPEG), methoxypolypropylene glycol, copolymers of polyethylene glycol and methoxy polypropylene glycol, dextran, and polylactic-polyglycolic acid, polyethylene glycol-diacid, PEG monoamine, MPEG monoamine, MPEG hydrazide, MPEG imidazole, copolymers of polyethylene glycol, methoxypolypropylene glycol, or mixtures thereof.

28. The fluorochrome conjugate of claim 29, wherein the solubility enhancing group is polyethylene glycol (PEG).

29. The fluorochrome conjugate of claim 4, wherein the photosenstizers are chlorins.

30. The fluorochrome conjugate of claim 4, wherein the chlorins are chlorin e6.

31. The fluorochrome conjugate of claim 4, wherein the photosensitizers are porphyrins.

32. The fluorochrome conjugate of claim 4, wherein the photosenstizers are independently rose bengal, bacteriochlorin, hematoporphyrin, chlorin e6, tetraphenylporphyrin, porfimer sodium, or benzoporphyrin.

33. The fluorochrome conjugate of claim 4, further comprising a solubility enhancing group, wherein at least one photosensitizer associates with the solubility enhancing group to form a photosensitive moiety.

34. The fluorochrome conjugate of claim 33, wherein the photosensitizer is a chlorin and the solubility enhancing group is polyethylene glycol (PEG), methoxypolyethylene glycol, methoxypolypropylene glycol, copolymers of polyethylene glycol and methoxypolypropylene glycol, dextran, polylactic-polyglycolic acid, or mixtures thereof.

35. The fluorochrome conjugate of claim 34, wherein the photosensitive moiety comprises chlorin e6 and polyethylene glycol.

36. The fluorochrome conjugate of claim 33, wherein the photosensitizer is rose bengal or a porphyrin and the solubility enhancing group is polyethylene glycol (PEG), methoxypolyethylene glycol, methoxypolypropylene glycol, copolymers of polyethylene glycol and methoxypolypropylene glycol, dextran, polylactic-polyglycolic acid, or mixtures thereof.

37. The fluorochrome conjugate of claim 1, wherein the at least two fluorochromes are near-infrared fluorochromes.

38. The fluorochrome conjugate of claim 1, wherein the at least two fluorochromes have excitiation and emission maxima in range of about 500 nm to about 900 nm.

39. The fluorochrome conjugate of claim 1, wherein the at least two fluorochromes are a combination of photosenstizer fluorochromes and non-photosensitizer fluorochromes.

40. The fluorochrome conjugate of claim 1, wherein the at least two fluorochromes are a combination of photosenstizer fluorochromes and quencher.

41. The fluorochrome conjugate of claim 4, further comprising at least one targeting moiety.

42. A fluorochrome conjugate comprising,
a dendrimer;
a peptide comprising a protease cleavage site;
a solubility enhancing group;
at least two fluorochromes, each covalently linked by a protease cleavage site to the dendrimer at positions; and
a spacer compound, wherein the spacer compound links a fluorochrome to the dendrimer.

43-56. (canceled)

57. A fluorochrome conjugate comprising:
a polylysine dendrimer;
a PEG solubility enhancing group; and
at least two chlorin e6 molecules covalently linked through a spacer to the dendrimer at optical-quenching positions, wherein the spacer comprises β-alanine and a cathepsin S enzymatic cleavage site.

58. A method of treating a subject having a disorder characterized by unwanted cellular proliferation, the method comprising:
(a) administering the fluorochrome conjugate of claim 1 to a subject;
(b) allowing the fluorochrome conjugate to distribute within the subject; and
(c) illuminating the fluorochrome conjugate with light of a wavelength sufficient to produce cytotoxic singlet oxygen.

59-63. (canceled)

64. A method for selectively imaging two different target cells of a subject simultaneously, the method comprising:
(a) administering to a subject one or more fluorochrome conjugates of claim 1, wherein said at least two fluorochomes emit distinct wavelengths of light upon illumination;
(b) allowing said one or more fluorochrome conjugates to distribute within the subject;
(c) illuminating the subject with light of a wavelength sufficient to be absorbed by the fluorochromes of said one or more fluorochrome conjugates; and
(d) detecting the optical signals emitted by said fluorochromes.

65. A method of treating a subject having a disorder characterized by unwanted cellular proliferation, the method comprising:
(a) administering to a subject a fluorochrome conjugate of claim 1, wherein at least one fluorochrome is a photosenstizer having optical properties distinct from the other fluorochrome(s);
(b) allowing the fluorochrome conjugate to distribute within the subject;
(c) illuminating the subject with light of a wavelength sufficient to be absorbed by the other fluorochrome(s) of the fluorochrome conjugate;
(d) detecting an optical signal emitted by the other fluorochrome(s);
(e) illuminating the subject with a second light of a wavelength sufficient to produce cytotoxic singlet oxygen by the photosensitizer; and
(f) detecting fluorescence emitted by the photosensitizer.

66. (canceled)

67. (canceled)

68. A kit for treating a subject having a disorder characterized by unwanted cellular proliferation comprising one or more unit dosage forms of one or more fluorochrome conjugates of claim 1 and instructions for use.

69. (canceled)

70. (canceled)

71. A kit for imaging a target cell or cells in a subject comprising one or more unit dosage forms of one or more fluorochrome conjugates of claim 1 and instructions for use.


SEQUENCE LISTING (7 sequences)

SEQ ID NO: 1 - protein, 9 aa, Artificial Sequence
  Description of Artificial Sequence Synthetic peptide
GPICFFRLG

SEQ ID NO: 2 - protein, 7 aa, Artificial Sequence
  Description of Artificial Sequence Synthetic peptide
HSSKLQG

SEQ ID NO: 3 - protein, 5 aa, Artificial Sequence
  Description of Artificial Sequence Synthetic peptide
PICFF

SEQ ID NO: 4 - protein, 6 aa, Artificial Sequence
  Description of Artificial Sequence Synthetic peptide
HSSKLQ

SEQ ID NO: 5 - protein, 6 aa, Artificial Sequence
  Description of Artificial Sequence Synthetic peptide
PXGXAG

SEQ ID NO: 6 - protein, 10 aa, Artificial Sequence
  Description of Artificial Sequence Synthetic peptide
GVVQASCRLA

SEQ ID NO: 7 - protein, 4 aa, Artificial Sequence
  Description of Artificial Sequence Synthetic peptide
ALRA